(19) United States
(12) Patent Application Publication
(10) Pub. No.: US 2025/0115834 A1
(43) Pub. Date: Apr. 10, 2025

(54) COMPOSITIONS COMPRISING GRAPE SKIN EXTRACTS AND METHODS OF PREPARATION AND USE THEREOF

(71) Applicant: PHANTASM LIMITED, Hataitai, Wellington (NZ)

(72) Inventor: Joshua RUFFELL, Hataitai, Wellington (NZ)

(73) Assignee: PHANTASM LIMITED, Hataitai, Wellington (NZ)

(21) Appl. No.: 18/716,842

(22) PCT Filed: Nov. 11, 2022
(86) PCT No.: PCT/NZ2022/050142

§ 371 Date: Jun. 5, 2024

(30) Foreign Application Priority Data
Dec. 9, 2021 (NZ) 783291
Dec. 9, 2021 (NZ) 783293

(51) Int. Cl. C12C 11/00; C12C 12/00

(52) U.S. Cl. CPC C12C 11/003; C12C 12/006; C12C 12/008

(57) ABSTRACT

The present disclosure encompasses compositions prepared from grape skin extracts, as well as chemical compounds prepared from these skins. Also encompassed are methods of preparing these compositions and compounds, and methods of using these compositions and compounds. Particularly encompassed are methods of using the compositions and compounds for preparing various beverages, including fermented beverages, such as beers.

### RELATED APPLICATIONS

This application claims the benefit of New Zealand patent application 783291, filed on 9 Dec. 2021, and New Zealand patent application 783293, filed on 9 Dec. 2021, both of which are incorporated by reference herein in their entirety.

### FIELD

The present disclosure relates to compositions prepared from grape skin extracts, and chemical compounds prepared from grape skin extracts. The present disclosure relates also to methods of preparing such compositions and compounds, and methods of using such compositions and compounds, including methods of preparing beverages, including beers and other fermented drinks.

### BACKGROUND

The production of fermented beverages is intrinsically a biotechnological process. The conversion of raw materials into a fermented beverage relies on microbial activity and many different enzymatic reactions.

Traditionally, beer is made from four key ingredients: malted cereals (barley or other), water, hops, and yeast. Cider is traditionally made from fruit juice and yeast, while kombucha is traditionally produced from fruit juice combined yeast and bacterial cultures. Yet, there is also a movement away from traditional fermentation ingredients and methods, and a trend towards more modem techniques.

The worldwide beer, cider, and kombucha industries have grown at an astonishing rate over the past decade, with the number of new manufacturers rapidly increasing. Microbreweries and craft breweries, in particular, have become increasingly numerous.

However, the widespread availability of craft products also means substantial competition between the various producers, and there is a considerable demand for new and improved consumables. Given this, there is a pressing need for new compositions and new methods for preparing fermented beverages.

### SUMMARY

In one aspect, the present disclosure encompasses a method for preparing a composition comprising a grape skin extract, the method comprising:
(i) obtaining skin of white wine grapes, such skin having flesh and juice components substantially removed,
(ii) freezing the skin of the white wine grapes,
(iii) thawing the skin of the white wine grapes,
(iv) incubating the skin of the white wine grapes in aqueous solution at about 45° C. to about 75° C.,
(v) obtaining a liquid extract produced by the incubating of the skin of the white wine grapes,
(vi) optionally removing sugars from the liquid extract,
(vii) optionally drying the liquid extract to produce a substantially dried product.

In various aspects:

The white wine grapes are aromatic white wine grapes.

The white wine grapes are selected from the group consisting of: white wine grapes from New Zealand, Australia, South Africa, South America, and any combination thereof.

The white wine grapes are selected from the group consisting of: Sauvignon Blanc, Albariño, Chenin Blanc, Colombard, Friulano, Tocai, Grüner Veltliner, Traminer, Verdicchio, Verdejo, Vermentino, Scheurebe, Maccabeo, Gewürztraminer, Riesling, Muscat, Petit Manseng, Pinot Gris, and Tokay grapes, and any combination thereof.

The white wine grapes comprise Sauvignon Blanc grapes.

The white wine grapes consist essentially of Sauvignon Blanc grapes.

The white wine grapes are Sauvignon Blanc grapes.

The substantially removing of (i) comprises crushing or pressing.

The pressing is performed by a pneumatic press or a basket press.

The pressing is performed with a press setting ranging from about 600 to about 800 litres per tonne.

The pressing is performed with a press setting ranging from about 650 to about 720 litres per tonne.

The method includes addition of at least one antioxidant or preservative before or after (i).

The at least one antioxidant is a sulphite.

The at least one antioxidant is ascorbic acid.

The aqueous solution of (i) is water or a solution consisting essentially of water.

The freezing of (ii) comprises blast freezing.

The freezing of (ii) comprises cryomaceration.

The incubating of (iv) comprises incubation at about 50° C. or higher, or about 55° C. or higher, or about 60° C. or higher, or about 65° C. or higher, or about 70° C. or higher.

The incubating of (iv) comprises incubation at about 50° C. to about 70° C.

Optionally, the liquid extract of (v) is contacted with further grape skin processed with any of (i)-(iii) to obtain a double extraction liquid extract.

Optionally, the double extract liquid is contacted with further grape skin processed with any of (i)-(iii) to obtain a triple extraction liquid extract.

The liquid extract of (v) is pasteurised.

The liquid extract of (v) is pasteurised by high pressure pasteurisation.

The removing of sugars of (vi) is by column absorption or desorption.

The removing of sugars of (vi) is by fermentation.

The drying of (vii) comprises freeze drying.

The composition comprises a milled form.

The composition comprises a pelleted form.

The composition is mixed with one or more liquids to produce a liquid or semi-liquid form.

The composition comprises a flake or powder.

The powder is prepared with a sieve size of about 4 mm to about 2 mm.

The composition comprises a liquid form or a semi-liquid form.

The composition is combined one or more yeast.

The composition is combined one or more bacteria.

The composition is combined one or more enzymes.

The one or more yeast are selected from the group consisting of: Saccharomyces, Brettanomyces, Kloeckera, Candida, Hanseniaspora, and Pichia yeast.

The one or more yeast include a Pichia kluyveri yeast.

The one or more yeast include a Saccharomyces cerevisiae yeast.

The Saccharomyces cerevisiae yeast is a brewer's yeast.

The one or more yeast include yeast used for conversion of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include a bacteria used for lactic acid production,

The one or more enzymes include β-lyase.

The one or more enzymes include β-glucosidase.

The composition is combined with one or more hops or hops component(s).

The composition is combined with lupulin.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

In one other aspect, the present disclosure encompasses a composition comprising a grape skin extract, the composition being prepared according to a preceding aspect.

In various aspects:

The composition comprises a milled form.

The composition comprises a pelleted form.

The composition comprises a liquid or semi-liquid form.

The composition comprises a flake or powder.

The composition is combined with one or more yeast.

The composition is combined with one or more bacteria.

The composition is combined with one or more enzymes.

The one or more yeast are selected from the group consisting of: Saccharomyces, Brettanomyces, Kloeckera, Candida, Hanseniaspora, and Pichia yeast.

The one or more yeast include a Pichia kluyveri yeast.

The one or more yeast include a Saccharomyces cerevisiae yeast.

The Saccharomyces cerevisiae yeast is a brewer's yeast.

The one or more yeast include a yeast used for conversion of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include bacteria used for lactic acid production.

The one or more enzymes include β-lyase.

The one or more enzymes include β-glucosidase.

The composition is combined with one or more hops or hops component(s).

The composition is combined with lupulin.

The composition is formulated to include one or more hops.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

In yet one other aspect, the present disclosure encompasses a composition comprising a grape skin extract and at least one additional ingredient, the composition being prepared according to a preceding aspect.

In various aspects:

The composition comprises a milled form.

The composition comprises a pelleted form.

The composition comprises a liquid or semi-liquid form.

The composition comprises a flake or powder.

The at least one additional ingredient is one or more yeast.

The at least one additional ingredient is one or more bacteria.

The at least one additional ingredient is one or more enzymes.

The at least one additional ingredient is one or more hops or hops component(s).

The at least one additional ingredient is lupulin.

The one or more yeast are selected from the group consisting of: Saccharomyces, Brettanomyces, Kloeckera, Candida, Hanseniaspora, and Pichia yeast.

The one or more yeast include Pichia kluyveri yeast or a Saccharomyces cerevisiae yeast.

The Saccharomyces cerevisiae yeast is a brewer's yeast.

The one or more yeast include a yeast used for conversion of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include bacteria used for lactic acid production.

The one or more enzymes include β-lyase.

The one or more enzymes include β-glucosidase.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

In one further aspect, the present disclosure encompasses a method of making a fermented beverage, comprising:
introducing a composition comprising a grape skin extract during a fermentation and/or prior to initiation of a fermentation,
wherein the composition comprising the grape skin extract is prepared according to a preceding aspect, and
carrying out the fermentation, thereby obtaining the fermented beverage.

In various aspects:

The composition is further introduced post-fermentation.

The composition comprises a milled form.

The composition comprises a pelleted form.

The composition comprises liquid or semi-liquid form.

The composition comprises a flake or powder.

The method includes introducing one or more yeast during the fermentation and/or prior to initiation of the fermentation.

The method includes introducing one or more bacteria during the fermentation and/or prior to initiation of the fermentation.

The method includes introducing one or more enzymes during the fermentation and/or prior to initiation of the fermentation.

The method includes introducing one or more hops or hops components during the fermentation, prior to initiation of the fermentation, and/or after the fermentation.

The composition includes one or more yeast.

The composition includes one or more bacteria.

The composition includes one or more enzymes.

The composition includes one or more hops or hops components.

The one or more yeast are selected from the group consisting of: Saccharomyces, Brettanomyces, Kloeckera, Candida, Hanseniaspora, Pichia yeast, and any combination thereof.

The one or more yeast include a Pichia kluyveri yeast.

The one or more yeast include a Saccharomyces cerevisiae yeast.

The one or more yeast include a brewer's yeast.

The one or more yeast include yeast used for conversion of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include a bacteria used for lactic acid production.

The one or more hops components include lupulin.

The one or more hops components include hops terpenes.

The one or more hops components include hops esters.

The one or more enzymes include β-lyase.

The one or more enzymes include β-glucosidase.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

The fermented beverage is an alcoholic, non-alcoholic, or reduced-alcohol beverage.

The fermented beverage is a beer.

The fermented beverage is selected from the group consisting of: barley or other cereal beers, maize beers, millet beers, oat beers, rice beers, rye beers, sorghum beers, and wheat beers, and/or

The fermented beverage is selected from the group consisting of: ales, lagers, pilsners, porters, saisons, and stouts.

The beer is an India Pale Ale beer.

The beer is a New England IPA.

The beer is a hazy IPA.

The fermented beverage is a beer-wine hybrid.

The fermented beverage is a fruit beer.

The fermented beverage is a cider.

The fermented beverage is a kombucha.

In yet a further aspect, the disclosure encompasses a fermented beverage prepared using the method according to a preceding aspect.

In various aspects:

The fermented beverage is an alcoholic, non-alcoholic, or reduced-alcohol beverage.

The fermented beverage is a beer.

The fermented beverage is selected from the group consisting of: barley or other cereal beers, maize beers, millet beers, oat beers, rice beers, rye beers, sorghum beers, and wheat beers.

The beer is selected from the group consisting of: ales, lagers, pilsners, porters, saisons, and stouts.

The beer is an India Pale Ale beer.

The beer is a New England IPA.

The beer is a hazy IPA.

The fermented beverage is a beer-wine hybrid or a fruit beer.

The fermented beverage is a cider.

The fermented beverage is a kombucha.

The foregoing brief summary broadly describes the features and technical advantages of certain embodiments of this disclosure. Further technical advantages will be described in the detailed description and examples that follow. Novel features that are believed to be characteristic of the disclosed subject matter will be better understood from the detailed description when considered in connection with any accompanying figures and examples. However, the figures and examples provided herein are intended to help illustrate this subject matter or assist with developing an understanding of this subject matter, and are not intended to limit its scope.

This disclosure refers specifically to New Zealand provisional application 765291, and International patent application PCT/NZ2021/050090, which are incorporated by reference herein in their entirety.

### BRIEF DESCRIPTION OF THE DRAWINGS

FIG. 1A: Grape skins in solvent before filtration.

FIG. 1B: Filtrate and cake after pressing.

FIG. 1C: Before (Left) and after (Right) centrifugation.

FIG. 1D: Grape skins after extraction and decanting.

FIG. 1E-1G: Liquid extracts after processing by reverse osmosis.

FIG. 2: The concentration of thiol precursors as the extraction temperature increases with and without seeds for varying extraction times. No seeds (NS), and with seeds (S). 50a indicates samples having 50° C. incubation and addition of ascorbic acid (0.01% w/w).

FIG. 3: The concentration of key polyphenols as the extraction temperature increases with and without seeds. The seed polyphenols start to be extracted at 70° C. 50a indicates samples having 50° C. incubation and addition of ascorbic acid (0.01% w/w).

FIG. 4: The concentration of key flavonoids as the extraction temperature increases with and without seeds. 50a indicates samples having 50° C. incubation and addition of ascorbic acid (0.01% w/w).

FIG. 5: Freeze dried permeate (Left) and freeze dried retentate (Right).

FIG. 6A: Schematic for specific extraction method in one aspect of this disclosure.

FIG. 6B: Schematic for specific extraction method in one aspect of this disclosure.

FIG. 7: Schematic for specific extraction method in one aspect of this disclosure.

FIG. 8: Schematic for specific extraction method in one aspect of this disclosure.

FIG. 9: Sample analysis. Graph showing total solids recovered from the supernatant and the levels of thiol precursors in the supernatant (Cys-3SH and GSH-3SH). R1 and R2 at 50° C., and R3 and R4 at 70° C.

FIG. 10: Calculated levels for each sample. Graph showing total solids recovered from each extraction and the levels of thiol precursors from each extraction (Cys-3SH and GSH-3SH). R1 and R2 at 50° C., and R3 and R4 at 70° C.

FIG. 11: Graph showing polyphenolic levels for the extracts. R1 and R2 at 50° C., and R3 and R4 at 70° C.

FIG. 12: Solution in tank after clarification membrane.

FIG. 13: Flux curve for the reverse osmosis process on grape skin extract.

FIG. 14: Sensory data obtained from beer prepared using OYL-011 yeast, with addition of no extract (control; upper panel), or R3 extract (middle panel), or R4 extract (lower panel).

FIG. 15: Sensory data obtained from beer prepared using OYL-402 yeast, with addition of no extract (control; upper panel), or R3 extract (middle panel), or R4 extract (lower panel).

FIG. 16: Sensory data obtained from beer prepared using OYL-405 yeast, with addition of no extract (control; upper panel), or R3 extract (middle panel), or R4 extract (lower panel).

FIG. 17: Comparative sensory data obtained from beer prepared using OYL-011, OYL-402, or OYL-405 yeast, with addition of no extract (control).

FIG. 18: Comparative sensory data obtained from beer prepared using OYL-011, OYL-402, or OYL-405 yeast, with addition of R3 extract.

### DETAILED DESCRIPTION

The following description sets forth numerous exemplary configurations, parameters, and the like. It should be recognised, however, that such description is not intended as a limitation on the scope of what is disclosed, but is instead provided as a description of exemplary embodiments.

All references, including patents and patent applications, cited in this specification are hereby incorporated by reference. No admission is made that any reference constitutes prior art. Nor does discussion of any reference constitute an admission that such reference forms part of the common general knowledge in the art, in New Zealand or in any other country.

### Definitions

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “consisting essentially of”, as used herein, may refer to the presence of a component in a composition. For example, an extract may be at least 80% by weight of the composition, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by weight of the composition (% w/w). For liquids, an extract may be at least 80% by volume of the composition volume, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by volume of the composition volume (% v/v).

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, levels of compounds or concentration levels, as described in detail herein. In particular, the term “about” encompasses a 10% to 15% deviation (positive and negative) in the stated value or range, particularly 10% deviation (positive and negative) in the stated value or range.

As used herein, “beer” refers to a fermented beverage prepared from cereal grains, or roots, or other plant parts. Different types beers may be flavoured with one or more types of hops. Exemplary beers include but are not limited to various cereal beers (e.g., beers prepared from malted barley), maize beers, millet beers, oat beers, rice beers, rye beers, sorghum beers, and wheat beers, and any combination of these, as well as various ales, lagers, pilsners, porters, radlers, saisons, and stouts. Beer-wine hybrids are also included, that is, beers brewed in combination with one or more wine grapes. Further included are fruit beers, for example, beers brewed in combination with one or more fruits. Other beers are described in detail herein.

“Concentrate” for example, in relation to a grape skin, refers to remaining material where the flesh and juice components of the grape have been partly or substantially removed. In addition to this, seeds and/or stems may be partly or substantially removed. Removal of the flesh and juice components may be by one or more of crushing, pressing, drying, and other means. A concentrate may be prepared as a particular composition, for example, as a paste, flake, cake, tablet, powder, plug, or pellet. A concentrate may be prepared by extraction methods. Various preparations and methods for obtaining these are set out further below.

An “extract”, for example, in relation to a grape skin, refers to material obtained by incubation of the grape skin with one or more solvents. Solvents may be aqueous or non-aqueous. Solvent extraction may be carried out by incubation in one or more of: water, alcohols (e.g., methanol, ethanol), acetone, acids (e.g., tartaric acid, citric acid), and salts (e.g., natural deep eutectic solvents), and any combination of these. Solutions that consist essentially of water may be utilised as solvents. An extract composition may comprise, for example, as a paste, flake, cake, tablet, powder, plug, or pellet form. The composition may comprise a liquid or semi-liquid (e.g., paste, gel) form. Details of various preparations for grape skin extract compositions are provided herein.

Utilised herein “fermented beverage” refers to consumable liquids prepared by yeast fermentation. These include but are not limited to beverages such as beers, beer-wine hybrids (classified as a type of beer), fruit beers, and ciders. Kombuchas are also included. The fermented beverage may be an alcoholic beverage, a non-alcoholic beverage, or a reduced-alcohol beverage. In certain circumstances, wines are specifically excluded as fermented beverages.

As noted herein “freeze drying” refers to a lyophilisation procedure. It will be understood that the terms “freeze drying” does not exclude the use of higher temperatures (i.e., higher than freezing temperatures). For example, higher than freezing temperatures may be used for removing residual moisture during the secondary drying phase for freeze drying procedures.

“Grape” as used herein encompasses any fruit of the genus Vitus, and any hybrid, cultivar, variety, and genetic derivative thereof. Encompassed, specifically, are wine grapes, including Vitis vinifera, and particularly, white wine grapes, including aromatic white wine grapes, such as the particular varietal grapes of Sauvignon wines, including Sauvignon Blanc wines, as well as the varietal grapes of Albariño, Chenin Blanc, Colombard, Friulano (e.g., Tocai), Grüner Veltliner, Traminer, Verdicchio, Verdejo, and Vermentino wines. Also included are Scheurebe, Maccabeo, Gewürztraminer, Riesling, Muscat, Petit Manseng, Pinot Gris, and Tokay wine grapes. Other grapes are described in detail herein. It will be understood that one or more different grapes (or grapes from different regions) may be combined and used. [00200]“Hops” when referring to a beverage additive refers to seed cones (also called flowers or strobiles) of the genus Humulus, and any hybrid, cultivar, variety, and genetic derivative thereof. Specifically encompassed are hops of Humulus lupulus, and particularly, H. 1. var. lupulus, H. 1. var. lupuloides (e.g., H. americanus), H. 1. var. cordifolius, H. 1. var. pubescens, and H. 1. var. neomexicanus. Exemplary hops include but are not limited to American hops and New Zealand hops, as well as hops from Australian, English, French, Belgian, German, South African, or other sources. Hops of interest are set out in this specification, including specific cultivars. It will be understood that one or more different hops (or hops from different regions) may be combined and used. In addition, wet or dry hops may be utilised, or combinations thereof. Particularly noted are whole leaf hops and components obtained from hops, for example, one or more hops extracts, hops powders, hops pellets, hops plugs, and the like. Cryogenically prepared hops are specifically included, i.e., hops cones that are frozen, for example, by liquid nitrogen, and have the lupulin glands separated from the hops leaf bract. Other exemplifications are provided herein.

A “genetic derivative” of a plant refers to offspring, sports, or other cultivars that are obtained from the parent stock. This includes offspring obtained from a genetic cross with the parent, e.g., F1 progeny or F2 progeny.

“Sauvignon Blanc” grapes refer to the specific white wine grapes of Vitis vinifera originating from Western France in the Loire Valley and Bordeaux wine regions, but also grown in notable regions of South Africa, Chile, New Zealand, California, Iran, and the Ukraine. “Sauvignon Blanc” is alternatively known as Sauvignon Jaune, Fumé Blanc, Blanc Fume, Feigentraube, and Muskat-Silvaner, amongst other names.

“Sulphur compounds” refers to various chemical agents associated with aroma and/or flavour in fermented beverages. These include but are not limited to volatile thiols and their precursors (i.e., thiol precursors), as well as furanthiols, mercaptans, esters, phenols, and terpenoids.

“Thiols” refer to sulphur compounds that are analogs of an alcohol. Particular thiols of interest include volatile thiols associated with grapes and/or hops. These include but are not limited to 3-mercaptohexan-1-ol (3MH), 3-mercaptohexyl acetate (3MHA), 4-methyl-4-mercaptopentan-2-one (4MMP), 4-mercapto-4-methylpentan-2-ol (4MMPOH), 3-sulfanyl-4-methylpentan-1-ol (3S4MP), 3-sulfanyl-4-methylpentyl acetate (3S4MPA), 3-mercapto-3-methylbutan-1-ol (3MMB), 3-mercapto-2-methylpropanol (3MMP). Various precursor compounds for volatile thiols are also included (i.e., thiol precursors), for example, S-3-(hexan-1-ol)-L-cysteine (Cys-3MH/C3MH), S-4-(4-methylpentan-2-one)-L-cysteine (Cys-4MMP/C4MMP), S-3-(hexan-1-ol)-glutathione (Glut-3MH/G3MH) and S-4-(4-methylpentan-2-one)-glutathione (Glut-4MMP/G4MMP) for 3MH and 4MMP. In relation to wine grapes, varietal thiols and non-varietal thiols are specifically noted.

As used herein, the term “yeast” refers to one or more strains used for fermentation. Exemplary yeast strains include but are not limited to Saccharomyces, Pichia, Brettanomyces, Kloeckera, Candida, and Hanseniaspora strains. Specifically noted for fermentation are Pichia kluyveri strains and Saccharomyces cerevisiae strains. Other strains are set out herein.

As used herein, the term “bacteria” refers to one or more strains used for fermentation. Exemplary bacterial strains include but are not limited to those of the order Lactobacillales, including, for example, Lactobacillus, Lactococcus, Leuconostoc, Streptococcus, Pediococcus, Oenococcus, Weissella strains. Specifically noted for fermentation are Lactobacillus delbrueckii, Lactobacillus plantarum, Pediococcus damnosus strains. Other strains are set out herein.

### Compositions Comprising Grape Skin Extracts and Associated Chemical Compounds

The applicant has found that grape skin extracts may be prepared and used as beneficial compositions for preparing fermented beverages, such as beers, beer-wine hybrids, fruit beers, and ciders, and also kombuchas. In particular, it has been demonstrated that the skins from white wine grapes, and particularly, Sauvignon Blanc varietal grapes, can be processed to obtain extract compositions useful for enhancing desirable aromas and flavours in fermented beverages.

Grapes, and specifically wine grapes, have been shown to contain sulphur compounds that contribute positively to beverage aromas. Notable amongst these are compounds identified as volatile thiols. See, e.g., Coetzee et al, 2012; Roland et al., 2011; Lund et al, 2009; Ribéreau-Gayon et al, 2006; Tominaga et al, 1998. Volatile thiols are therefore of particular interest for the compositions and methods of the present disclosure. Non-limiting examples of these are set out in Table 1, below.

Typically, grapes have low levels of volatile thiols, and instead contain the corresponding precursor compounds. It is during the fermentation process that yeast convert the various precursor compounds into the volatile thiols (i.e., biotransformation takes place), and thereby impart aromas for the final beverage. Non-limiting examples of precursors are set out in Table 2, below.

The three main volatile thiols associated with tropical nuances in wine are 3MH, 3MHA, and 4MMP. 4MMP precursors are found mainly in juice and flesh of grapes. In contrast, 3MH precursors are retained in the skin, particularly when grapes are processed under cooler temperatures and with gentler pressing. With this in mind, the present application proposes methods of preparing and extracting grape skins while optimising levels of volatile thiols and precursors in these compositions. These compositions, in turn, are useful for enhancing fermented beverages as described in detail herein.

Notably, beers produced with certain hops are also known to contain volatile thiols. Non-limiting examples of these are set out in Table 3, below. Beer hops are also known to produce terpenoids, particularly sulphur-containing terpenoids, which are useful for imparting aromas and flavours. See, e.g., Holt et al., 2019. Therefore, the present disclosure encompasses compositions comprising the grape skin extracts, as described herein, which may be combined with various hops compositions. Also encompassed are methods of using the various compositions in fermentation. This is set out in detail further below.

Thus, the present disclosure relates generally to a composition prepared from grape skin extraction. Particularly noted are grape skins from wine grapes. Of specific note are grape skins from white wine grapes. While specific compositions prepared from white wine grapes are described herein, that is, Sauvignon Blanc grapes, it is understood that the present disclosure is readily adapted to use with different types of grapes. Amongst exemplary grapes are those that exhibit particular biochemical, taste, and/or aroma similarities to Sauvignon Blanc wine grapes. These include but are not limited to Albariño, Chenin Blanc, Colombard, Friulano (e.g., Tocai), Grüner Veltliner, Traminer, Verdicchio, Verdejo, and Vermentino, wine grapes. Other white wine grapes are encompassed, such as those with noted volatile thiol components. These include but are not limited to Chardonnay, Gewürztraminer, Gros Manseng, Koshu, Maccabeo, Muscat, Muscadet, Petit Arvine, Petit Manseng, Pinot Blanc, Pinot Gris, Riesling, Scheurebe, Semilion, Sylvaner, and Tokay wine grapes. Red wine grapes with noted volatile thiol components are also encompassed. Such include but are not limited to Cabernet wine grapes, such as Cabernet Franc and Cabernet Sauvignon, and Carignan, Grenache, Malbec, Merlot, Pinot Noir, and Syrah (also known as Shiraz) wine grapes. Further noted are: Airén, Carmenere, Gamay, Petite Syrah, Petit Verdot, Sangiovese, Tempranillo Ugni Blanc, Viognier, and Zinfandel wine grapes. Any combination of the above may also be used.

In one particular aspect, the composition is prepared from Vitis vinifera Sauvignon wine grapes, for example, Sauvignon Blanc wine grapes, or one or more of the other grapes described herein. In other aspects, one or more genetic derivatives from this plant/these plants may be used. For example, it may be desirable to use F1 or F2 progeny from a genetic cross that includes the parent stock of the plant. Alternatively, any sports or other cultivars obtained from the parent may be used. It may be desirable to source the grapes from Europe, for example, from France or Germany; from America, for example, from California; from Australia; from New Zealand; from South Africa; or from South America, for example, from Chile. A combination of grapes from different sources may also be used.

The composition is preferably formulated to comprise a grape skin extract. It will be understood that the extract is prepared from the liquid remains of the grapes after crushing or pressing. The extract will encompass components derived from the skins and, optionally, the seeds and/or stems of the grapes. Accordingly, the composition may be prepared in dried or semi-liquid form. The composition may be formulated, for example, as a flake, powder, pellet, plug, tablet, capsule, syrup, suspension, gel, pomace, puree, paste, or as drops. The composition may be provided in sachet form, for example, a powder or liquid sachet.

In certain aspects, it may be desirable to formulate the grape skin extract composition into a dried product. Noted formulations include flakes, pellets, plugs, tablets, and capsules. The capsules may include powdered contents. The powder may be provided in free flowing form or as a solid cake. The composition may be provided as a powder for forming a suspension, powder for forming a solution, bulk granules, or bulk powder. In certain circumstances, the dried product may be combined with one or more liquids to re-constitute as a liquid or semi-liquid composition. Other formulations are also possible, as described herein.

TABLE 1
Key volatile thiol compounds from wine grapes

| Key compound | Aroma(s) | Structure |
|---|---|---|
| 3-mercaptohexan-1-ol (3MH) (C₆H₁₄OS) also called 3-sulfanylhexan-1-ol (3SH) | passion fruit, grapefruit, gooseberry, guava | 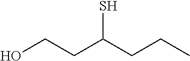 |
| 3-mercaptohexyl acetate (3MHA) (C₈H₁₆O₂S) also called 3-sulfanylhexyl acetate | passion fruit, grapefruit, box tree, gooseberry guava | 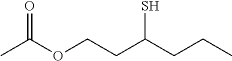 |
| 3-mercapto-3-methylbutan-1-ol (3MMB) also called 3-methyl-3-sulfanylbutan-1-ol | cooked leeks | 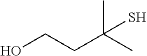 |
| 3-mercapto-2-methylpropanol (3MMP) | broth, sweat | 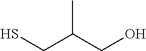 |
| 4-methyl-4-mercaptopentan-2-one (4MMP) (C₆H₁₂OS) also called 4-methyl-4-sulfanylpentan-2-ol (4MSP) | box tree, passion fruit, broom, blackcurrant, cat urine | 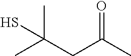 |
| 4-mercapto-4-methylpentan-2-ol (4MMPOH) (C₆H₁₄OS) also called 4-methyl-4-sulfanylpentan-2-ol | citrus zest | 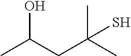 |

TABLE 2
Key volatile thiol precursors

| Precursor | Structure |
|---|---|
| 3MH precursor (E)-hex-2-enal | 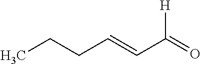 |
| 3MH precursor Glutathionylated precursor (G3MH; also GSH-3SH) | 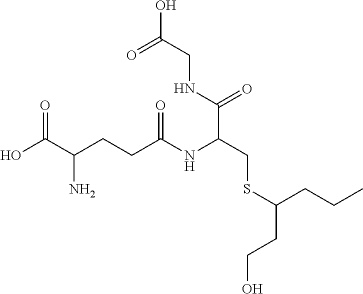 [SMILES: CCCC(CCO)SCC(NC(=O)CCC(N)C(=O)O)C(=O)NCC(=O)O] |
| 3MH precursor Cysteinylated precursor (Cys3MH/C3MH; also Cys-3SH) | 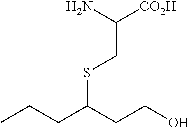 |
| 4MMP precursor Glutathionylated precursor (G4MMP) | 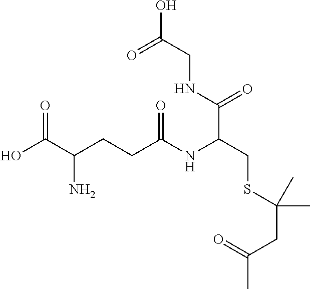 [SMILES: CC(=O)CC(C)(C)SCC(NC(=O)CCC(N)C(=O)O)C(=O)NCC(=O)O] |
| 4 MMP precursor Cysteinylated precursor (Cys4MMP/C4MMP) | 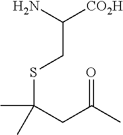 |
| 3MHA precursor 3-mercaptohexan-1-ol (3MH) | 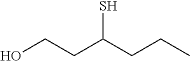 |

TABLE 3
Key volatile thiol compounds from hops

| Key compound | Aroma(s) | Structure |
|---|---|---|
| 3-mercaptohexan-1-ol (3MH) (C₆H₁₄OS) also called 3-sulfanylhexan-1-ol (3SH) | passion fruit, grapefruit, gooseberry, guava | 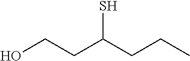 |
| 3-sulfanylpentan-1-ol (3SP) | citrus zest and grapefruit | 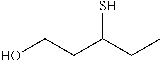 |
| 3-sulfanyl-4-methylpentan-1-ol (3S4MP) | grapefruit and/ or rhubarb | 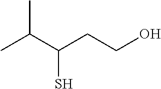 |
| 3-sulfanyl-4-methylpentyl acetate (3S4MPA) | grapefruit, peach, and/or rhubarb | 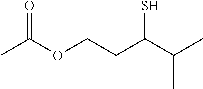 |
| 4-methyl-4-mercaptopentan-2-one (4MMP) (C₆H₁₂OS) also called 4-methyl-4- sulfanylpentan-2-one (4MSP) | box tree, passion fruit, broom, blackcurrant, cat urine | 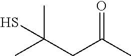 |

As particular examples, the composition of the present disclosure may be combined with one or more hops or one or more ingredients obtained from hops, e.g., one or more hops components. For example, the composition may be formulated as a powder, pellet, or plug, and may include a combination of grape skin extract along with hops, hops extract, and/or hops powder. Alternatively, the composition may include a grape skin extract combined with whole leaf hops or wet hops. Specifically noted as a hops component is lupulin. As examples, lupulin can be utilised in powder or pellet form, or other dried form. Lupulin enriched hops pellets are specifically noted, e.g., Type 45 (John I Hass). In addition, concentrated lupulin may be provided in the form of cryogenically prepared hops, as described herein. Alternatively, liquid form(s) of lupulin may be utilised, and in the same way liquid form(s) of the grape skin extract composition may be utilised. Particularly noted are liquid hops products, e.g., Incognito® and Spectrum® (John I Hass), and pourable lupulin fractions. Therefore, the grape skin extract composition in any form may be combined with any form of lupulin to produce a mixture.

As various exemplifications, lupulin can be obtained from one or more hops sources, such as American, Australian, English, German, New Zealand, or South African hops. New Zealand hops, such as Nelson Sauvin, are noted. Particular lupulin compositions include but are not limited to: Citra lupulin powder or pellets (e.g., LupuLN2®), Altus™ lupulin pellets, Amarillo® lupulin pellets, Bravo™ lupulin pellets, Caltus lupulin pellets, Calypso™ lupulin pellets, Cascade lupulin pellets, Centennial lupulin pellets, Comet lupulin pellets, Lemondrop® lupulin pellets, Lotus lupulin pellets, and Sultana™ lupulin pellets. As further exemplifications, CO₂ extractions and ethanol extractions of hops which produce liquid forms of lupulin may be utilised. Extractions that include hop terpenes (e.g., monoterpenes) are specifically noted. Hops oils are additionally noted. Cryogenically prepared hops (e.g., Cryo Hops® from Yakama Chief Hops), which include a concentrated source of lupulin, are of particular interest. Also of interest are Hopsteiner lupulin pellets and Lupomax® hops pellets from John I Hass.

It will be understood that one type of hops may be used for the disclosed combinations, or different types of hops may be used together. The hops may be obtained from one or more sources, such as American, Australian, English, German, New Zealand, or South African hops. New Zealand hops, such as Nelson Sauvin, are particularly useful. Exemplary hops include but are not limited to: Hallertau Blanc, Tomahawk, Simcoe, Summit, and Cascade hops, as well as Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Saaz-CZ, Talus, Vista, Ella, Enigma, and Vic Secret hops. Various hop blends may be used, for example, Cryo Pop™ blends such as TRI2304CR blended hops (see, e.g., Yakima Chief Hops). The detailed hop variety information will be known and readily identified. See, e.g., International Hop Growers Convention Hop Variety Codes, such as the IHGC hop variety list available from usahops.org; see, also, https://www.yakimachief.com/commercial/hop-varieties.html?product_list_limit=all

The composition may contain one or more additional ingredients, for example, one or more binders, disintegrants, flavours, colours, sweeteners, flow agents, anti-caking agents, sorbents, including one or more antioxidants/preservatives as described herein. Exemplary ingredients include but are not limited to: stearin, magnesium stearate, and stearic acid; saccharides and their derivatives, e.g., disaccharides: sucrose, lactose; polysaccharides and their derivatives, e.g., starches, cellulose or modified cellulose such as microcrystalline cellulose and cellulose ethers such as hydroxypropyl cellulose; sugar alcohols such as isomalt, xylitol, sorbitol and maltitol; proteins such as gelatin; synthetic polymers such as polyvinylpyrrolidone, polyethylene glycol; fatty acids, waxes, shellac, and plant fibres, e.g., corn protein zein; hydroxypropyl methylcellulose; crosslinked polymers, e.g., crosslinked polyvinylpyrrolidone (crospovidone), and crosslinked sodium carboxymethyl cellulose (croscarmellose sodium); sodium starch glycolate; silicon dioxide, fumed silica, talc, and magnesium carbonate.

In certain aspects, the composition of the present disclosure may include a significant level of grape skin extract. As exemplifications, the percentages of extract in the composition may range from at least 75% to at least 95% grape skin extract; or at least 85% to at least 95% grape skin extract; or at least 90% to at least 95% grape skin extract in the composition (w/w or v/v). The composition may consist essentially of grape skin extract as described herein. Where combined with hops (or component(s) obtained from hops, for example, hops powder or hops extract), the percentages of extract in the composition may range from at least 15% to at least at least 45% grape skin extract; or at least 25% to at least 45% grape skin extract; or at least 35% to at least 45% grape skin extract in the composition (w/w or v/v). Similarly, the percentages of hops or hops component(s) in the combination may range from at least 15% to at least at least 45% grape skin extract; or at least 25% to at least 45% grape skin extract; or at least 35% to at least 45% grape skin extract in the composition (w/w or v/v).

It is expected that the composition of the present disclosure will include one or more of the beneficial sulphur compounds from wine and/or hops. As noted, volatile thiol compounds are of particular interest for the composition, and specifically, 3MH, 3MHA, and 4MMP, and their precursors. As examples, the composition may include concentrations of one or more volatile thiols (e.g., one or more of 3MH, 3MHA, 4MMP, and/or their precursors) ranging from at least 2 to at least 500 ng/kg, at least 10 to at least 1000 ng/kg; or at least 40 to at least 800 ng/kg; or at least 100 to at least 700 ng/kg; or at least 500 to at least 600 ng/kg; or concentrations of at least 50 ng/kg, at least 100 ng/kg, at least 500 ng/kg, at least 1000 ng/kg, at least 5000 ng/kg, or at least 10,000 ng/kg. As further examples, concentrations may be ranging from at least 2 to at least 500 ng/L, at least 10 to at least 1000 ng/L; or at least 40 to at least 800 ng/L; or at least 100 to at least 700 ng/L; or at least 500 to at least 600 ng/L; or concentrations of at least 50 ng/L, at least 100 ng/L, at least 500 ng/L, at least 1000 ng/L, at least 5000 ng/L, or at least 10,000 ng/L. In certain circumstances, it may be desirable to supplement the sulphur compounds present in the composition. In particular, purified sulphur compounds may be introduced to the composition, including purified volatile thiols such as one or more of 3MH, 3MHA, and 4MMP, and/or their precursors.

In relation to this, it will be understood that other known assays may also be used to analyse the disclosed compositions, and the present disclosure is not limited to one particular assay for bioactive compounds. Components suitable for analysis include but are not limited to sulphur compounds, for example, volatile thiols and their precursors, as well as phenolics, antioxidants, carbohydrates, etc. It will be understood also that the levels of components can be determined for solid forms of the extracts and also can be readily determined for liquid forms of the extracts. Exemplary methods include but are not limited to column chromatography, gas chromatography, e.g., gas chromatography-mass spectrometry (GC-MS), liquid chromatography, e.g., high-performance liquid chromatography (HPLC), ultra-performance liquid chromatography tandem mass spectrometry (UPLC-MS/MS), spectrophotometry, e.g., FTIR spectrophotometry, and affinity chromatography.

It has already been noted that the composition of the present disclosure finds particular use for preparing fermented beverages. Methods of preparing and using the disclosed compositions are set out in detail below.

### Methods of Producing Compositions Comprising Grape Skin Extracts

This disclosure relates generally to a composition prepared from grape skin extraction. Particularly noted are grape skins from wine grapes. Of specific note are compositions prepared using grape skins from white wine grapes, such as Sauvignon Blanc wine grapes. Yet, it will be understood that various types grapes and compositions obtained therefrom are also encompassed by the present disclosure.

As an exemplification of the preparation process, grape skins may be obtained by crushing and/or pressing grapes, and partly or substantially removing the juice and flesh from the grape skins. For example, the practitioner may obtain grape skins (e.g., source or produce), such grape skins having juice and flesh partly or substantially removed. The obtained grape skins can then be frozen or chilled. For example, blast freezing or blast chilling may be used. Individual quick freezing (IQF) may be used. If frozen, the grape skins may be thawed. Following this, the grape skins can be incubated in a solvent, for example, an aqueous solution such as water, or an aqueous solution consisting essentially of water. The liquid extract can be separated from the remaining skins materials. In certain aspects, the liquid extract can be treated to substantially remove sugars in the extract. In further aspects, the extracted liquid can be treated by pasteurisation, for example, high pressure pasteurisation.

In further aspects, the liquid extract can be substantially dried to produce a dried product. Drying aids may be utilised as described herein. As an example, freeze drying may be used alone or in combination with other drying methods. This dried product, in turn, be further processed to produce a flaked, powdered, or pelletised product. In various aspects, the grape skin extract may be prepared such that the solvent is incubated with the grape seeds and/or stems, or alternatively, the method can include one or more steps to remove grape seeds and/or stems. The drying after the separation step may be by evaporation, for example, air drying may be used. The drying may be by centrifugation under vacuum. Reverse osmosis or forward osmosis may be utilised. In certain circumstances, heating may be avoided or minimised during the separation and drying steps.

In certain circumstances, it may be useful to estimate thiol precursor levels in the grapes in advance of processing, and target specific growing blocks and/or harvest windows. In initial preparatory stages, the grapes may undergo a pre-treatment process which may include the well known steps of ripening, inspecting, grading, and/or sorting of the grapes. With regard to ripening, it is preferable to use ripe or mature grapes when producing the compositions. Ripeness can be assessed using widely known and used methods in the art. For example, relevant measurements for ripe grapes may range from about 21.7° Bx/12 degree Baumé/93° Oe to about 27.1° Bx/15 degree Baumé/119° Oe. For ripe grapes, particular Brix measurements may be at least about 22° Bx, at least about 23° Bx, at least about 24° Bx, at least about 25° Bx, at least about 26° Bx, or at least about 27° Bx. As exemplifications, grapes may be harvested at a range of about 21 to about 22° Bx. As an alternative, ripeness may correlate to a titratable acidity level between about 0.60-0.99% to about 0.65-0.95%. As a further alternative, ripeness may correlate to a Brix to TA ratio from about 30:1 to about 35:1. Ripeness can be measured prior to picking or processing the grapes. In certain circumstances, overripened grapes may be used.

As part of the processing, the grapes or grape skins may be cleaned and/or sterilised. The grapes may be passed through an assembly having one or more roller brushes for removing dirt or other foreign matter. Conventional washing techniques may be employed. For example, a series of spray nozzles may be used. Wash additives aiding cleansing or reducing the bacteria count may be employed according to local regulations and requirements. As exemplifications, the grapes may be washed by a chlorine wash and/or an ozone impregnated water wash followed by a freshwater rinse.

For processing, the grapes may be conveyed into a hopper. This can be tapered to form a funnel to direct the grapes for pressing. Pressing may be performed with or without a machine. Machine pressing may be accomplished by vertical style pressing, e.g., basket press devices, or horizontal style pressing, e.g., airbag or bladder devices. Other devices include membrane presses, moving head presses, screw presses, impulse presses, and belt presses. Tank presses may also be used. Pressing may be adapted to be performed continuously or in batches. It may be desirable to remove stems prior to pressing by a destemmer/crusher. Alternatively, whole cluster pressing can be used, where the grapes are pressed while still attached to the stems.

The pressing methods may be adapted to apply different levels of force to the grapes as needed. See, e.g., Maggu et al., 2007. As exemplifications, pressures of about 0.4 atm to about 2.4 atm; or about 0.8 atm to about 2 atm; or about 1 atm to about 1.5 atm; or about 0.4 atm, about 0.8 atm, about 1 atm, about 1.2 atm, about 1.4 atm, about 1.8 atm, about 2 atm, about 2.2 atm, or about 2.4 atm may be used for pressing. Pressing may utilise a press setting that ranges from about 500 to about 900 litres per tonne, or about 600 to about 800 litres per tonne, or about 710 to about 760 litres per tonne, or about 740 to about 760 litres per tonne. For example, settings of at least 740, at least 745, at least 750, at least 755, or at least 760 per tonne may be used. Noted settings are at about 720 to about 750 litres per tonne, or about 720, about 730, about 740, about 750, or about 760 litres per tonne.

Pressing may be performed, for example, by a pneumatic press or a basket press. In certain aspects, processing can be relatively gentle (“soft pressing”) compared to conventional pressing techniques. For example, with soft pressing, e.g., pressing set at 650 to 720 litres per tonne or less, there is reduced fragmentation of the skin. In particular, it may be desired that only a minor proportion (generally less than 20%) of skin is fragmented by this process. As an exemplification, a gentle press cycle may be used with an inert gas to avoid oxidisation of the material. Amongst available commercial presses, Bucher Inertys systems may be used, e.g., Bucher XPlus 22 to 80 or Bucher XPert 100 to 450. Computerised systems may be used to control the exact pressure being applied to the grapes and the number of cycles applied. See, e.g., J. Robinson (ed) The Oxford Companion to Wine Third Edition, 2006, pages 285-286, 545-546, 767.

To ensure minimal degradation of ingredients, the preparation process (e.g., pressing) may be performed at a temperature of less than 20° C. In various embodiments, the process is performed at a temperature ranging from −4° C. to 20° C.; or from −1° C. to 15° C.; or from 1° C. to 12° C.; or at about 0° C., about 1° C., about 2° C., about 3° C., about 4° C., about 5° C., or about 6° C. These temperatures may be kept during the preparation process, including the storage of the grapes, prior to being broken open, and during the crushing or pressing process. For example, these temperatures may be kept at least from the point that the grapes have been broken open. Use of such temperatures can reduce oxidation of the grapes and the minimise the use of reducing agents.

Alternatively, or along with these reduced temperature, antioxidants or other preservatives may be added to the grapes or grape skins to retain freshness. As exemplifications, sulphites may be used, e.g., sulphur dioxide. Other examples include but are not limited to sorbic acid, sodium sorbate, potassium sorbate, potassium metabisulphite, citric acid, ascorbic acid, malic acid, tartaric acid, propionic acid, and benzoic acid, for example, in the form of its sodium salt, e.g., sodium benzoate. Any combination of antioxidants/preservatives may be used. Specifically noted are combinations of sulphur dioxide and ascorbic acid. Noted also are combinations of potassium metabisulphite and ascorbic acid. The grape skin extract may include, for example, less than 1% of a preservative, e.g., about 0.005% to about 0.5%, or about 0.05% to about 0.15%, or may include about 0.04%, about 0.06%, about 0.08%, about 0.1%, about 0.12%, about 0.14%, about 0.16%, about 0.18%, or about 0.2% of a preservative (w/w).

As specific exemplifications, sulphur dioxide may be added to achieve levels ranging from about 25 to about 55 ppm, or about 30 to about 50 ppm, or about 35 to about 45 ppm in the crushed or pressed grape skins. Preferably, sulphur dioxide levels are less than 300 mg/kg. As other exemplifications, potassium metabisulphite may be added at about 50 to about 70 g per tonne of grapes or pressed grapes, or at about 55 to about 70 g per tonne, or at about 65 to about 68 g per tonne. As further exemplifications, ascorbic acid may be added at about 15 to about 45 parts per tonne of grapes or pressed grapes, or at about 20 to about 35 parts per tonne, or at about 25 to about 35 parts per tonne.

In certain aspects, the antioxidant(s) or other preservative(s) is/are added prior to pressing or crushing, or at least within 10-20 minutes of pressing or crushing. It is also possible to combine antioxidant or preservative agents to increase overall antioxidant activity. To further reduce oxidation, it is possible to use de-oxygenation and inert gas purging. In particular, carbon dioxide and/or nitrogen gas may be introduced during the pressing or crushing process. For example, nitrogen flushing may be used during pressing. It is also possible to add one or more fining agents before, during, or after the pressing or crushing process to protect sulphur compounds. For example, one or more animal or soil components, or one or more synthetic components, may be used as fining agents, including casein, skim milk, egg white (e.g., egg albumin), gelatin, isinglass, bentonite, carbon, and polyvinylpolypyrrolidone (PVPP). In particular aspects, copper may be specifically avoided.

In some circumstances, it may be desirable to adjust the pH of the grape skins or that of the final composition to approximate levels used in fermentation. In particular, it may be useful to obtain a pH range from about 5.0 to about 7.0; or about 5.0 to about 6.5; or about 5.0 to about 6.0; or about 5.0 to about 5.5; or a pH of about 5.1, about 5.2, about 5.3, about 5.4, about 5.5, about 5.6, about 5.7, about 5.8, about 5.9, or about 6.0.

Specific pre-treatments may be utilised. For example, the grape skins may be treated by ultrasonic vibrations and/or pulsed electric field processing. These treatments may be performed prior to or in lieu of the chilling and/or freezing steps as described herein.

In particular aspects, the grape skins are chilled or frozen directly after removal of the juice and flesh to maintain freshness. Chilling or freezing may also be carried out within 1-2 hours or within 12-24 hours of removal, as needed. Blast chilling and blast freezing are particularly useful methods. As exemplifications, chilling temperatures may range from about 1° C. to about 12° C., about 2° C. to about 10° C., about 4° C. to about 6° C., or may be about 5° C., while freezing temperatures may range from about −20° C. to about 0° C., or about −15° C. to about −5° C., or about −18° C. to about −8° C., or may be about −10° C. For example, a must chiller may be set to about 11° C. to about 13° C. Other methods utilise 3600 freezing or chilling and/or ACVCS next generation freezing technology. Once frozen or chilled, the skins can be stored under these conditions until required. Frozen grape skins can be thawed for drying or may be dried directly by freeze drying.

In certain circumstances, cryomaceration may be used as a chilling or freezing methodology. For example, following crushing or pressing, the grape skins may be exposed to low or cold temperatures for more than 1 hour. Exemplary temperatures include about −18° C. to about 0° C., or about −15° C. to about 0° C., or about −10° C. to about 0° C., or about −5° C. to about 0° C., or about 4° C. to about 15° C., about 5° C. to about 15° C., about 6° C. to about 12° C., about 8° C. to about 12° C., or about 10° C. The duration may be dependent on the temperature, for example, from about 2 hours up to 7-14 days. The cooling may utilise cryogens/coolants such as solid carbon dioxide (dry ice), liquid carbon dioxide, or nitrogen. Notably, the cold shock from cryomaceration produces a larger volume of water in the grape skins and the weight of the skins helps to break up the cellular membrane (cell cracking).

In a particular exemplification, grapes may be quickly frozen and then slowly thawed. For example, the grapes may be frozen to about −30° C. to about −10° C., or about −25° to about −15° C., or to about −20° C. (−4 F), using one or more cryogens, such as dry ice, and then thawed, for example, over about 18 hours, over about 20 hours, over about 24 hours, over about 36 hours, or over about 48 hours. In particular aspects, the process may be carried out so as to maximise ice crystals formation, to damage the grape skin tissue, and enhance levels of beneficial sulphur compounds such as volatile thiols and their precursors. It is possible to utilise an extraction process with an aqueous solution, e.g., water or a solution consisting essentially of water, for the thawed grape skins. See, for example, Jelley et al., 2016; Jelley et al., 2020.

As one specific option, the grapes may be pressed, frozen, thawed, and then incubated in one or more aqueous solutions (e.g., water) which is/are able to act as a solvent. Alternatively, the grapes may be pressed, chilled, removed from the chill, and then incubated in one or more aqueous solutions (e.g., water). The solvent(s) may then be separated and further processed. For example, evaporation can be used to dry down the solvent(s) (e.g., a rotary evaporator may be used). The water may also be treated by filtration prior to evaporation. For example, one or more of pad filters, mesh filters, centrifuge filters, or other brewing filters may be used. Nanofiltration methods are specifically noted. Reverse osmosis and similar methods (e.g., membrane permeation processes) may be used to concentrate the components in the extract. The dried down and/or filtered product (e.g., paste product) can be further dried and milled as described below. Without wishing to be bound by theory, it is believed that the soaking process may assist with extraction of Cys-3SH and GSH-3SH thiol precursors from the grape skins.

Successive incubations with the same solvent or different solvents may be used. For example, water and/or solutions consisting essentially of water may be utilised as described herein. Incubations in solvent(s) may be carried out at room temperature or at elevated temperatures. Cooler temperatures may also be used. For example, incubations may be performed at about 30° C. to about 90° C., or about 40° C. to about 80° C., or about 45° C. to about 75° C., or about 50° C. to about 80° C., or about 50° C. to about 70° C., or about 45° C. to about 55° C., or about 65° C. to about 75° C., or about 50° C. or higher, or about 55° C. or higher, about 60° C. or higher, or about 65° C. or higher, about 70° C. or higher, or about 75° C. or higher, or about 80° C. or higher. As further examples, incubations may be performed at about 5° C. to about 10° C., or about 5° C. to about 15° C., or about 5° C. to about 20° C., or about 5° C. to about 25° C., or about 5° C. to about 30° C. Other ranges are also included, for example, 5° C. to about 90° C., or about 10° C. to about 80° C., or about 15° C. to about 75° C., or about 20° C. to about 75° C., or about 25° C. to about 75° C.

Incubations may be carried out over different time periods. For example, incubations may be performed at about 20 to about 120 minutes, or about 30 to about 90 minutes, or about 40 to about 80 minutes, or about 40 to about 50 minutes, or at least 45 minutes. Following or during the incubation with the solvent(s), it may be helpful to include a further pressing step. This may be useful in releasing further beneficial components from the grape skins. In some circumstances, pre-treatment steps may be utilised prior to incubation with solvent(s). For example, the grape skins may be treated by ultrasonic and/or possibly pulsed electric field (PEF) treatment. In various aspects, the ratio of solvent(s) to skins may be about 1 to about 6 (e.g., about 1:6), or about 1 to about 5.5. (e.g., 1:5.5), or about 1 to about 5 (e.g., about 1:5), or about 1 to about 4.5 (e.g., about 1:4.5) or about 1 to about 4 (e.g., about 1:4), or about 1 to about 3.5 (e.g., about 1:3.5), or about 1 to about 3 (e.g., 1:3) (w/w), or may range from about 1:6 to about 1:3, or about 1:5 to about 1:4, (w/w).

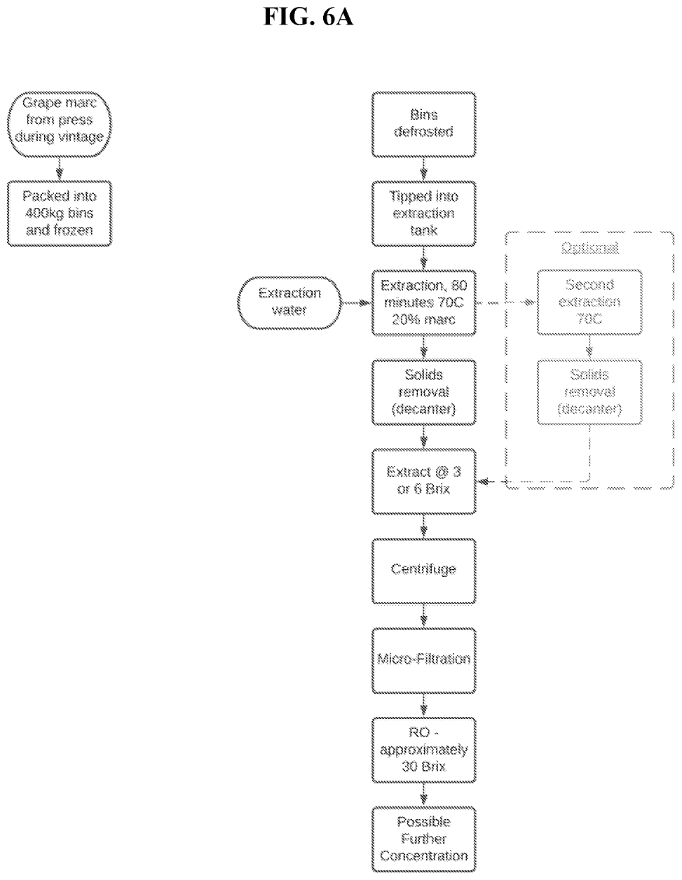

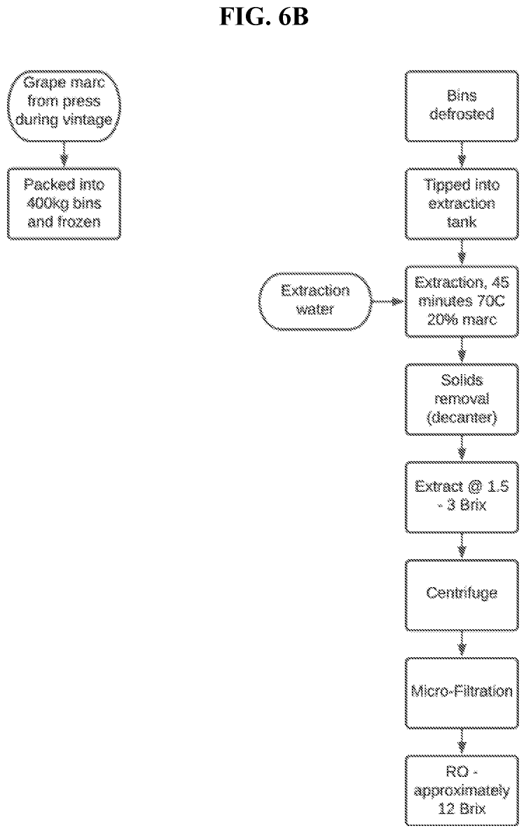

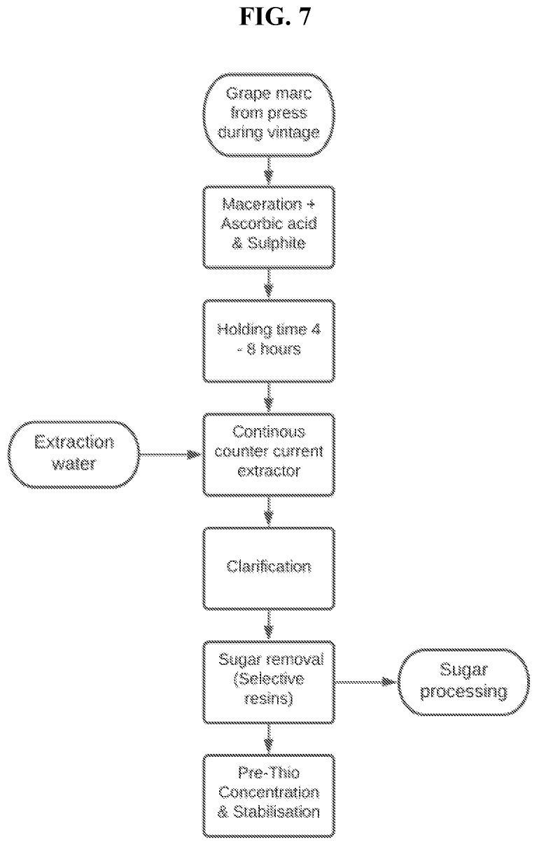

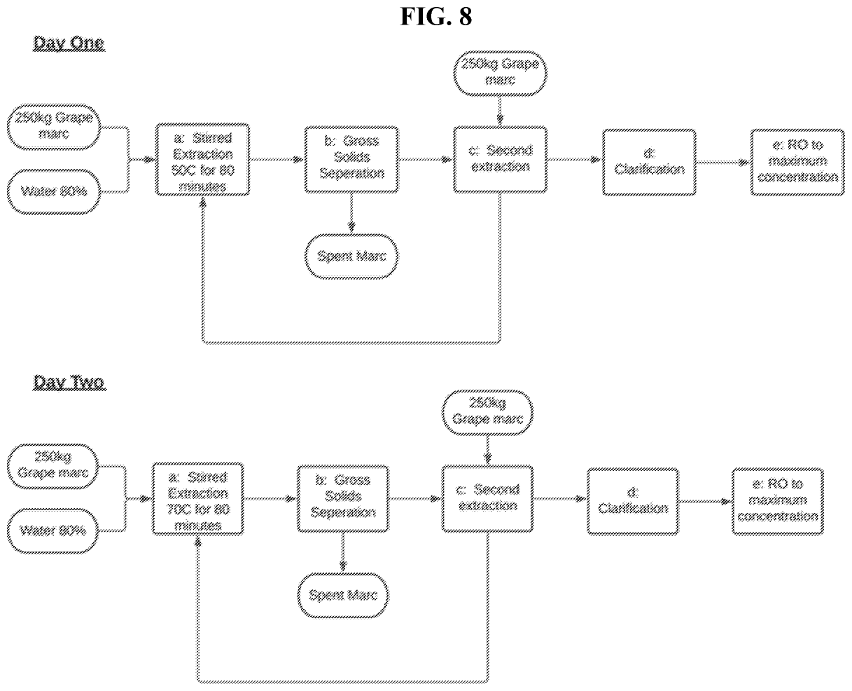

As one exemplification, grape skins may be pressed, and the resulting skins may be frozen. The grape skins (e.g., about 200 to about 400 kg) may be placed in a large tank containing water and/or other solvents. For example, various aqueous solutions may be utilised as solvents, including solutions consisting essentially of water. The ratio of water to skins may range from about 1:4 to about 1:5 (w/w), or about 1:4.5 to about 1:5.5 (w/w), or about 1:3 to about 1:6 (w/w). This water grape skin mixture can be stirred and incubated e.g., about 70° C., for various time periods, e.g., about 80 minutes. Solids may be removed by a decanter. The extract may be obtained at 3 to 6 Brix. Optionally, the extract may be centrifuged and microfiltered. As further options, the extract may be treated by reverse osmosis techniques, and may be further concentrated. As another option, a multiple extraction (e.g., double extraction) may be used, i.e., the liquid extract from the initial incubation may be used to incubate a further amount of grape skins. The resulting liquid extract may then be obtained and optionally centrifuged, filtered, concentrated, etc. (see, e.g., FIGS. 6-8).

As one other exemplification, the pressed grape skins (e.g., about 200 to about 400 kg) may optionally be macerated and incubated with one or more antioxidants, e.g., ascorbic acid and sulphite salt. The mixture may be incubated for various periods, e.g., about 2 to about 10 hours, or about 4 to about 8 hours. The skins may then be placed in a continuous counter current extractor, to which solvent(s) is/are also added. One or more other aqueous solutions may be utilised, e.g., water or solutions consisting essentially of water. The liquid extract may be removed and clarified. Optionally, the sugars may be removed from the liquid extract, e.g., by selective resins. By this or alternative means, the thiol precursors can be concentrated and stabilised. As a further option, the liquid extract may be subjected to pasteurisation. Exemplary methods for pasteurisation are described herein.

The liquid extract may be separated from the grape skins and further processed. For example, evaporation can be used to dry down the mixture (e.g., a rotary evaporator may be used). The liquid extract may also be treated by filtration prior to evaporation. For example, one or more of pad filters, mesh filters, centrifuge filters, or other brewing filters may be used. Reverse osmosis and/or other techniques for concentrating the extracted liquid may be used. In certain embodiments, forward osmosis may be used. An extraction product (e.g., liquid or paste product) can be dried and milled as described below. Thus, it can be advantageous for the extracted liquid to be substantially dried after separation from the grape skins.

In certain circumstances, it may be helpful to remove or substantially remove sugars from the grape skin extract composition. Various methods of absorption/desorption may be used. For example, column chromatography may be utilised. Exemplifications include high performance liquid chromatography columns, anion exchange columns (e.g., strong anion exchange columns), and solid phase extraction columns (e.g., polyamide columns, styrene-divinylbenzene columns). Other methods may be used, for example, fermentation methods whereby yeast or other microbes convert the sugars to alcohol.

In specific circumstances (e.g., when sugar removal is not performed), pasteurisation treatments may be utilised for the grape skin extract composition. For example, high pressure pasteurisation may be used, such as pasteurisation involving pressure levels of about 200 to about 400 MPa, or about 300 to about 600 MPa, or greater than 600 MPa. Such pressure levels may be employed in conjunction with elevated temperatures, for example, temperatures that are less than 40° C., less than 30° C., less than 20° C., less than 10° C., less than 5° C., or ranging from about 5° C. to about 40° C., or about 5° C. to about 30° C., or about 5° C. to about 20° C., or about 5° C. to about 10° C. Various high pressure processing methods (e.g., high hydrostatic pressure processing) may be adapted and applied as appropriate.

Drying can include freeze drying. Other drying methods may also be utilised. For example, evaporation may be used (e.g., vacuum drying). Methods involving drum drying may be used. In the drum-drying process, a paste may be dried at relatively low temperatures over rotating, high-capacity drums that produce sheets of drum-dried product. An additive may be used to accelerate or otherwise assist the drying process. For example, pea starch or other drying aids may be introduced. As further alternatives, belt drying or convection drying may be used. Any combination of drying methods may be employed.

Freeze drying techniques are widely known and commonly used. The freeze drying process may range from 5 to 60 hours; or 10 to 50 hours; or 15 to 40 hours; or about 20 to about 30 hours. Noted for freeze drying are ranges of about 23 to about 27 hours, or about 24 to about 26 hours. Importantly, freeze drying may be utilised to retain the beneficial compounds in the grape skins, for example, terpenes and key C₆ compounds, including volatile thiols and their precursors, as described in detail herein.

It may be desirable to use a particular freeze drying process for obtaining the dried product. See, e.g., de Torres et al., 2015. For example, a drying program may be used as part of an automated drying system. The process may include multiple drying steps, e.g., with step wise increases and reductions in temperature. Preferably, a primary drying setting is used for sublimation, followed by one or more secondary drying settings that are used to remove residual moisture. In particular aspects, the top temperature of the process does not exceed 35° C., or alternatively, the top temperature does not exceed 50° C. In other aspects, the temperature of the drying process ranges between −10° C. to 70° C. In one other aspect, at least 24 hours of dying is utilised. As specific exemplifications, chamber batch drying or continuous tunnel drying may be used, for example.

In certain aspects, an additive may be used to accelerate or otherwise assist the drying process. For example, pea starch or other drying aids may be utilised. Also noted as a drying aid is dextrin and various forms of dextrin, for example, maltodextrin. In addition, the dried products may be made to be cold water soluble or soluble in cold aqueous solutions. The resulting dried product may then be milled into a flake or powder form. Exemplary powders may have a sieve size of about 1 mm or less. Milling methods are well known and widely used in the art. Standard mesh sizes may be used to produce the powder, for example, US 20, US 23, US 30, US 35, US 40, US 45, or US 50 mesh sizes may be used. The sieve size for the powder may range from 8 mm to 0.3 mm; or 4 mm to 0.4 mm; or 4 mm to 1 mm; or may be about 4.0 mm, about 3.15 mm, about 3 mm, about 2.8 mm, about 2 mm, about 1 mm, about 0.84 mm, about 0.71 mm, about 0.59 mm, about 0.5 mm, about 0.47 mm, about 0.465 mm, about 0.437 mm, about 0.4 mm, about 0.355 mm, or about 0.3 mm. Powder may be packaged into nitrogen flushed bags to protect against oxidation, for example, 0.5 kg to 30 kg bags, or 1 kg to 20 kg bags.

It is expected that the composition of this disclosure will contain thiol precursors in sufficient quantities so as to provide a readily utilised source of such compounds. For example, the composition may include C3MH, at a concentration of at least 0.5 pg per gram, at least 0.8 μg per gram, at least 1 μg per gram, at least 1.2 μg per gram, at least 1.5 μg per gram, at least 1.8 μg per gram, at least 2 μg per gram, at least 2.2 μg per gram, at least 2.5 μg per gram, at least 3 μg per gram, at least 3.2 μg per gram, or at least 3.5 μg per gram, or a liquid equivalent thereof. The composition may include CG3MH at a concentration of at least 0.05 μg per gram, 0.08 μg per gram, at least 0.1 μg per gram, at least 0.12 μg per gram, at least 0.15 μg per gram, at least 0.18 μg per gram, at least 0.2 μg per gram, at least 0.22 μg per gram, at least 0.25 μg per gram, at least 0.27 μg per gram, at least 0.29 μg per gram, or a liquid equivalent thereof. The composition may include GC3MH at a concentration of at least 0.15 μg per gram, at least 0.16 μg per gram, at least 0.17 μg per gram, at least 0.18 μg per gram, at least 0.2 μg per gram, at least 0.25 μg per gram, at least 0.3 μg per gram, at least 0.35 μg per gram, at least 0.4 μg per gram, at least 0.45 μg per gram, at least 0.5 μg per gram, at least 0.55 μg per gram, at least 0.6 μg per gram, or at least 0.65 μg per gram, or a liquid equivalent thereof. The composition may include G3MH at a concentration of at least 2 μg per gram, at least 2.5 μg per gram, at least 2.8 μg per gram, at least 3 μg per gram, at least 3.2 μg per gram, at least 3.8 μg per gram, at least 4 μg per gram, at least 4.2 μg per gram, or at least 4.5 μg per gram, or at least 20 μg per gram, at least 50 μg per gram, at least 100 μg per gram, at least 150 μg per gram, at least 200 μg per gram, at least 250 μg per gram, at least 300 μg per gram, at least 400 μg per gram, or at least 500 μg per gram, or a liquid equivalent thereof.

It will be understood that the grape skin extract composition disclosed herein may be utilised on its own. In addition, as noted herein, the composition of the present disclosure may be combined with one or more hops or one or more components obtained from hops. As exemplifications, the composition that includes hops or hops component(s) may be formulated as a powder, pellet, or plug. Alternatively, the composition that includes hops or hops components may be liquid in form. In certain aspects, hops products may be prepared so as to be intermixed with grape skin extract. For example, grape skin extract (e.g., dry or liquid form(s)) may be added during preparation of hops pellets or hops plugs to produce a combined product, or grape skin extract powder may be added with hops powder to produce a combined product. In other aspects, hops or hops components may be modified with grape skin extract. For example, whole leaf hops, hops plugs, or hops/lupulin pellets (e.g., cryogenically prepared hops pellets) may be dipped, rolled in, sprinkled with, or dusted with grape skin extract to produce a combined product.

Combination products provide, for example, for hybrid pellets having one or more hops or hops components and grape skin components. These may have desirable levels of aromatic compounds, and may also have synergistic properties in the fermentation. Therefore, various combination products are set out herein and are encompassed by this disclosure. As particular examples, grape skin extract composition obtained from New Zealand Sauvignon Blanc wine grapes may be pelletised with one or more American, German, or English hops or hops components, or alternatively, Australian, New Zealand, or South African hops or hops components, or any combination of these. These exemplifications are provided as illustrations only, and other combinations are considered to be encompassed by this disclosure.

### Methods of Using Compositions Comprising Grape Skin Extracts

The applicant has found that compositions comprising grape skin extracts include chemical compounds that are useful for the preparation of fermented beverages such as beers, beer-wine hybrids, fruit beers, and ciders, as well as kombuchas. Particularly noted are the sulphur compounds such as volatile thiols, such as 3MH, 3MHA, and 4MMP, and their precursors. Therefore, it is expected that the beverages produced according to the present disclosure will include beneficial levels of sulphur compounds, including volatile thiols and their precursors.

As non-limiting exemplifications, the produced beverage may include at least 50 ng/L to at least 12,000 ng/L 3MHA; and/or at least 150 ng/L to at least 20,000 ng/L 3MH; and/or at least 5 ng/L to at least 100 ng/L 4MMP. For 3MH, in particular, levels may be at least 40 ng/L, at least 50 ng/L, at least 60 ng/L, at least 70 ng/L, at least 80 ng/L, at least 90 ng/L, at least 100 ng/L, at least 200 ng/L, at least 300 ng/L, at least 400 ng/L, at least 500 ng/L, at least 600 ng/L, at least 700 ng/L, at least 800 ng/L, at least 900 ng/L, at least 1000 ng/L, at least 1100 ng/L, at least 1300 ng/L, at least 1500 ng/L, at least 1700 ng/L, at least 1900 ng/L, at least 2000 ng/L, at least 2100 ng/L, or at least 10,000 ng/L. For 4MMP, levels may be at least 0.5 ng/L, at least 1 ng/L, at least 1.2 ng/L, at least 1.4 ng/L, at least 1.6 ng/L, at least 1.8 n/L, at least 2 ng/L, at least 2.2 ng/L, at least 2.4 ng/L, at least 2.6 ng/L, at least 2.8 ng/L, at least 3 ng/L, at least 4 ng/L, at least 6 ng/L, at least 15 ng/L, or at least 150 ng/L. For 3MHA, levels may be at least 2.5 ng/L, at least 5 ng/L, at least 6 ng/L, at least 7 ng/L, at least 8 ng/L, at least 9 ng/L, at least 10 ng/L, at least 11 ng/L, at least 12 ng/L, at least 15 ng/L, at least 20 ng/L, at least 30 ng/L, at least 50 ng/L, or at least 500 ng/L.

The beverage may also include one or more thiol precursors. For example, the beverage may include C3MH, at a concentration of at least 8 μg/L, at least 10 μg/L, at least 15 μg/L, at least 25 μg/L, at least 30 μg/L, at least 35 μg/L, at least 40 μg/L, at least 45 μg/L, at least 50 μg/L, at least 55 μg/L, or at least 60 μg/L, or a liquid equivalent thereof. As further examples, the beverage may include CG3MH at a concentration of at least 20 μg/L, 25 μg/L, at least 30 μg/L, at least 35 μg/L, at least 40 μg/L, at least 45 μg/L, at least 50 μg/L, at least 60 μg/L, at least 65 μg/L, at least 70 μg/L, at least 75 μg/L, at least 80 μg/L, at least 85 μg/L, at least 90 μg/L, at least 95 μg/L, or a liquid equivalent thereof. As further examples, the composition may include GC3MH at a concentration of at least 8 μg/L, at least 10 μg/L, at least 15 μg/L, at least 20 μg/L, at least 25 μg/L, at least 30 μg/L, at least 35 μg/L, at least 40 μg/L, at least 45 μg/L, at least 50 μg/L, at least 55 μg/L, or at least 60 μg/L, or a liquid equivalent thereof. As further examples, the composition may include G3MH at a concentration of at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, or at least 115 μg/L, or a liquid equivalent thereof.

The fermented beverage produced with the composition is expected to include increased levels of volatile thiols as compared to a beverage produced without the composition. For example, 3MH levels may be increased 1.5 fold to 15 fold, or at least 1.5 fold, at least 2 fold, at least 3 fold, at least 4 fold, at least 5 fold, at least 10 fold, at least 15 fold, or at least 20 fold. The 4MMP levels may be increased 2 fold to 20 fold, or at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 5 fold, at least 10 fold, at least 15 fold, at least 20 fold, or at least 25 fold. The 3MHA levels may be increased 1.2 fold to 12 fold, or at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 5 fold, at least 10 fold, at least 15 fold, or at least 20 fold.

For producing the beverages of the present disclosure, various organisms may be used. Brewer's yeasts that are used for fermentation are particularly noted. Exemplary yeasts include Saccharomyces yeasts, for example, Saccharomyces cerevisiae. Other yeasts include but are not limited to Brettanomyces, Candida, Hanseniaspora, Kloeckera, and Pichia yeasts, for example: B. bruxellensis, B. custersii, B. lambicus, B. intermedius, B. anomalus, C. stellata, C. zemplinina, C. pulcherrima, H. uvarum, H. osmophila, H. guilliermondii, K. apiculata, K. javanica, K. cortices, P. kluyveri, and S. pastorianus, S. uvarum, amongst others. Included also are Dekkera anomala, Naumovozyma dairenensis, and Debaryomyces spp. yeasts.

Yeast strains that are used for volatile thiol production (e.g., yeast suitable for biotransformation of thiol precursors into volatile thiols) are particularly noted, for example, S. cerevisiae and P. kluyveri strains. For kombucha, both yeast and bacterial cultures may be used, e.g., yeast such as a Zygosaccharomyces, Candida, Torulaspora, Pichia, Brettanomyces/Dekkera, Schizosaccharomyces, and/or Saccharomyces strains (as examples, Saccharomyces cerevisiae or Zygosaccharomyces kombuchaensis) along with bacteria, such as Komagataeibacter and/or Gluconobacter strains (as examples, Komagataeibacter xylinus or Komagataeibacter kombuchae). Combinations of any of the above organisms, including specific combinations with S. cerevisiae may also be used.

Exemplary strains include effective thiol producing strains, for example, strains that are used to convert thiol precursors into volatile thiols (e.g., 3MH or 4MMP), or strains that are used to produce the volatile thiols from other compounds. Other strains may convert 3MH to 3MHA, or produce other chemical conversions in relation to volatile thiol molecules. For example, various Saccharomyces and/or Pichia strains may be used for volatile thiol production. It is also possible to include yeasts (e.g., inactive yeasts) that produce glutathione, as a protection from oxidation. Yeast with increased R-lyase activity (e.g., increased expression levels and/or increased enzymatic function) and/or increased p-glucosidase activity (e.g., increased expression levels and/or increase enzymatic function) are specifically noted. Such yeast may be genetically engineered, for example, to include one or more mutations or chromosomal insertions. In addition to or as an alternative to this, such yeast may include one or more expression constructs, for example, one or more plasmids to provide R-lyase and/or P-glucosidase expression.

Particular P. kluyveri strains include, but are not limited to: PK-KR1, PK-KR2, (see, e.g., US 20170183612), and DSM 33235. Pichia kluyveri var kluyveri is specifically noted. Noted also is the commercially available product FROOTZEN® (Chr Hansen), which comprises P. kluyveri isolated from New Zealand grape juice. Particular S. cerevisiae strains include, but are not limited to: L2056, NT116, VIN7, VIN13, VL3, X5, and QA23 strains. Commercially available strains may be obtained, for example, from Wyeast, White Labs, Omega Yeast, Fermentis, Escarpment Laboratories, and other suppliers. Particular strains of interest include Wyeast 1318 London Ale III™ yeast, as well as Wyeast 1056 American Ale yeast; and also WLP066 London Fog Ale yeast, WLP518 Opshaug Kveik Ale yeast, and WLP077 tropicale yeast blend from White Labs; along with SafAle™ S-33, SafAle™ K-97, and SafAle™ S-04 from Fermentis; and OYL-061 Voss Kveik, or OYL-402 or OYL-405 yeast from Omega Yeast. Also noted are Berkeley Labs Tropics thiol yeast, Lallemand Lalbrew yeast, and other yeast with increased levels of β-glucosidase or β-lyase activity. Any mixture of yeast strains can be used to maximise production and biotransformation levels.

Notably, it is possible to obtain substantially increased levels of volatile thiols in beverages prepared using the composition of this disclosure in combination with yeast strains used for converting thiol precursor compounds to volatile thiols. For example, as compared to beverages prepared without the composition and without such yeast, levels of 3MH may be increased at least 30 fold, at least 35 fold, at least 40 fold, at least 45 fold, at least 50 fold, at least 60 fold, at least 70 fold, at least 80 fold, at least 90 fold, at least 100 fold, at least 120 fold, at least 180 fold, or at least 200 fold.

Bacterial strains that are used for lactic acid production are particularly noted, for example, strains of the order Lactobacillales. For example, lactic acid producing strains (i.e., lactic acid bacteria) may be utilised. These include, but are not limited to Lactobacillus, Lactococcus, Pediococcus, and Streptococcus strains. Specifically noted for fermentation are Lactobacillus acidophilus, Lactobacillus brevis, Lactobacillus buchneri, Lactobacillus delbruckii, Lactobacillus lactis, Lactobacillus plantarum, and Lactobacillus rhamnosus strains. Also noted are Pediococcus acidilactici, Pediococcus damnosus Pediococcus parvulus, Pediococcus pentosaceus strains. Commercially available lactic acid bacteria may be obtained, for example, from Wyeast, White Labs, Omega, and other suppliers. Particular strains of interest include WLP677, WLP672 from White Labs; 5335, 5223-PC, 4335 from Wyeast; OYL-605 from Omega; MIP-911, MIP-912, MIP-913, MIP-914 dein Propagate Lab; RVA 600 from RVA Yeast Labs. Other strains of interest include OYL-606 from Omega; MIP-920 from Propagate Lab; RVA 601 from RVA Yeast Labs; WLP661 from White Labs; 5733 from Wyeast. Any mixture of bacterial strains can be used to maximise lactic acid production and activity levels. It will be understood that mixtures of bacteria and yeast strains may also be utilised.

When preparing a beverage, the composition of this disclosure may be introduced together with the hops or hops component(s). Alternatively, introduction of the composition and the hops/hops component(s) may be separate. Introduction of the composition may be at one or more points during the beverage processing. The introduction of the hops/hops component(s) may be at one or more points during the beverage processing. For preparing fermented beverages, particular ratio amounts of the grape skin extract composition and the hops may be used. As exemplifications, ratio amounts of about 2.5 to about 1; about 2 to about 1; about 1 to about 1; about 1 to about 0.75; about 1 to about 0.5; about 1 to about 0.25 of grape skin extract composition to hops may be employed.

In other aspects, a combination of grape skin extract composition and hops may comprise or may consist essentially of about 10% to about 90% grape skin extract composition (w/w) and about 90% to about 10% hops (w/w), or about 15% to about 85% grape skin extract composition (w/w) and about 85% to about 15% hops (w/w), or about 20% to about 80% grape skin extract composition (w/w) to about 80% to about 20% hops (w/w), or about 20% to about 40% grape skin composition (w/w) to about 80% to about 60% hops (w/w), or about 25% to about 35% grape skin composition (w/w) to about 75% to about 65% hops (w/w).

The grape skin extract composition may be added when fermentation is already in progress. For example, the grape skin extract composition may be added to a wort stream at about 4 degrees Plato to about 8 degrees Plato, or at about 5 degrees Plato to about 7 degrees Plato. Upon addition of the grape skin extract composition, fermentation can then be continued, e.g., at temperatures of about 18° C. to about 22° C., or about 19° C. to about 21° C. As one specific option, the grape skin extract composition may be added before the start of fermentation, for example, during the boiling phase or whirlpool phase for brewing. It is also possible to add the grape skin extract composition after fermentation or proximate to the end of fermentation. In the same way, it is possible to add hops or hops component(s) after fermentation or proximate to the end of fermentation.

It will be understood that various methods may be combined or modified for beverage preparation, and the composition of this disclosure is not limited to use with particular methodologies. For example, where brewing is carried out, it will be possible to use mash hopping, first wort hopping, whirlpool hopping (e.g., hop stand), dry hopping, wet hopping, hop bursting, hop addition during the boil, hop addition proximate the end of the boil, or any combination of these methods. For dry hopping, it will be possible to employ dry hop addition in the primary fermenter and/or dry hop addition in the secondary fermenter (e.g., loose or contained). Hop addition (hopping) may be by introduction of one or more hops or hops component(s), or by combinations of one or more hops and hops component(s).

Notably, the disclosed methods may be utilised for enhancing or depressing thiol levels from fermentation, as needed. For example, it may be possible to promote higher volatile thiol levels (e.g., 3MH and/or 3MHA), by omitting dry hops or introducing these in reduced quantities. In the same way, it may be possible to promote more of a balance between increased volatile thiols and hop character, by introducing grape skin extract composition and hops in conjunction, for example, as a combined product, and/or via co-administration, such as with sequential or simultaneous addition.

As one exemplification, the grape skin extract composition may be added to the brewing process as a hot side addition. For example, the composition can be introduced into the mash tun and/or kettle/whirlpool. This would introduce concentrated thiol precursors, which could then be converted to volatile thiols during the fermentation stage. Specifically, this may be achieved by addition of a β-lyase enzyme and/or by fermentation with either a yeast strain used to boost volatile thiol compounds, for example, Cosmic Punch™ (OYL-402; Omega Yeast). Alternatively, or in addition to this, a standard strain of brewer's yeast could be used in combination with a co-fermentation strain, such as P. kluyveri. For example, the P. kluyveri could be added on day 1 of fermentation, and within the first 48 hours convert the thiol precursors into volatile thiol compounds. At this stage, a further standard strain of brewer's yeast could then be added to complete the fermentation. As a further option, dry hop additions may be made with further addition(s) of the grape skin extract composition to re-introduce thiol precursor, alongside the introduction of hops or hops component(s) (e.g., lupulin, terpenes, and/or esters) for combined fermentation effects.

In certain circumstances, it may be desirable to enrich the levels of beneficial sulphur compounds that are available during fermentation. In particular, purified sulphur compounds may be introduced prior to or during fermentation, including purified volatile thiols such as one or more of 3MH, 3MHA, and 4MMP, and/or their precursors. Purified compounds may also be introduced after fermentation, to supplement the levels of beneficial sulphur-containing components in the final beverage. It will be understood that the levels of sulphur compounds, for example, volatile thiols and their precursors, as well as phenolics, antioxidants, carbohydrates, etc, may be monitored before, during, and/or after fermentation. Various measurement methodologies can be used, as described here.

To maintain maximal levels of sulphur compounds, including the levels of volatile thiol compounds and their precursors, it is possible to use fining agents to negate metal catalysts and residual polyphenols during fermentation. Exemplary agents are noted herein. It is also possible to use de-oxygenation and inert gas purging during fermentation. For example, carbon dioxide and/or nitrogen gas may be introduced. In addition, it is possible to include one or more antioxidants/preservatives before, during, or after fermentation. Non-limiting examples of these compounds are provided herein.

One or more enzymes may also be used to optimise pressing and maximise aromatic compounds. For example, one or more pectolytic enzymes may be used. This includes commercially available enzymes such as those of LAFAZYM® PRESS. Also noted are aroma enhancing enzymes such as Rapidase® Expression Aroma enzymes and Lallemand Aromazyme™ enzymes. Noted as well are various lyase products, e.g., sulphur lyase such as Endozym® Thiol. Furthermore, it is possible to use higher fermentation temperatures (irrespective of yeast strain(s) used) to increase the release volatile thiols by yeast strains. For example, temperatures of at least 17° C., at least 18° C., at least 19° C., at least 20° C., at least 23° C., at least 25° C., at least 27° C., at least 30° C., at least 33° C., at least 35° C., or at least 37° C. can be used, for at least part of the fermentation period. In particular, Kveik yeast fermentations can be carried out from about 30 to about 37° C. It is also possible to reduce temperatures for the later stages of fermentation (e.g., reduction to about 15° C. to about 16° C.) to preserve the volatile compounds that have been released.

Following fermentation, the beverage will be ideally chilled and stored at low temperatures to minimise oxidation reactions, and also to reduce hydrolysis reactions converting 3MHA TO 3MH. It would therefore be preferable to keep the beverage at a temperature as low as possible without freezing during the time periods following fermentation, prior to bottling (or canning or kegging), and prior to consumption. Exemplary temperatures include but are not limited to temperature of less than 16° C., less than 15° C., less than 14° C., less than 12° C., less than 10° C., less than 8° C., less than 6° C., or less than 4° C. It is also preferably to carry out bottling, canning, or kegging as quickly as possible following fermentation, to reduce oxygen exposure.

It will be understood that the compositions of the present disclosure are useful for the preparation of various fermented beverages. Included amongst these are beers, such as beers made from cereals (e.g., barley malt), maize, millet, oats, rice, rye, sorghum, or wheat, or any combination of these. Specifically included are ales such as amber ales, Belgian style ales, blonde ales, brown ales, sour ales, wild ales, and also pale ales, such as American Pale Ale, India Pale Ale, and New Zealand Pale Ale; bocks, such as German bocks; lagers, such as pale lagers and dark lagers, as well as Vienna lagers; pilsners; porters, such as Baltic porters; radlers; saisons; session beers; and stouts, including Irish stouts and Imperial stouts. Particular ales of interest include Hazy IPAs, New England IPAs, Double IPAs, Triple IPAs, Sour IPAs, Fruit IPAs, Oat Cream IPAs, Milkshake IPAs, and Tropical IPAs. Other beers include weissbier, Berliner weisse, witbier, maibocks, English bitter, Biere de Garde, marzen, dunkel, dunkelweizen, and doppelbock beers.

Included also are various beer-wine hybrids, for example, beers brewed in combination with one or more types of wine grapes. White wine grapes are noted, in particular, for beer-wine hybrids. For example, Sauvignon Blanc grapes may be used or any other white wine grape, including those described herein, and any combinations of these. Also included are various ciders, such as dry ciders, and specifically including cloudy and clear ciders, and single and multiple fruit ciders, for example, ciders comprising juice from one or more of: crab apples, apples, pears, kiwifruits, and berries, such as strawberries, blackberries, raspberries.

Various fruit beers are further included, such as beers brewed with one or more fruits, for example, guavas, mangos, papayas, passionfruit, pineapple, plums, peaches, apricots, citrus, cherries, and berries, including Boysenberries, blueberries, raspberries, strawberries, and any combination of the preceding. Fruit may be introduced during beermaking via whole fruits, fruit parts, fruit juice, fruit purees, fruit extracts, or other means, and may act as an adjunct or flavouring for the beer. Kombuchas are included as well, e.g., kombuchas flavoured with one or more fruit juices and/or one or more spices. It will be understood that these are exemplifications, only, and other types of beers, beer-wine hybrids, fruit beers, ciders, and kombuchas will be known and well within the purview of the skilled person.

In various aspects, the composition of this disclosure may be utilised at appropriate levels to achieve the desired levels of thiol precursors and volatile thiol compounds. As exemplifications, the composition (e.g., powder form of the extract composition) may be introduced at amounts of at least 2 g/L, or at least 3 g/L or at least 4 g/L, or at least 5 g/L, or at least 6 g/L, or at least 7 g/L, or at least 8 g/L, or at least 9 g/L, or at least 10 g/L, or at least 11 g/L, or at least 12 g/L, or at least 13 g/L, or at least 14 g/L, or at least 15 g/L, or at least 16 g/L, or at least 17 g/L or at least 18 g/L, or at least 19 g/L, or at least 20 g/L, or ranging at about 3 g/L to about 18 g/L, or about 3 g/L to about 15 g/L, or about 3 g/L to about 12 g/L, or about 6 g/L to about 9 g/L, or about 7 g/L to about 8 g/L. As further exemplifications, composition (e.g., liquid form of the extract composition) may be introduced at amounts of at least 2 g/L, or at least 3 g/L or at least 4 g/L, or at least 5 g/L, or at least 6 g/L, or at least 7 g/L, or at least 8 g/L, or at least 9 g/L, or at least 10 g/L, or at least 11 g/L, or at least 12 g/L, or at least 13 g/L, or at least 14 g/L, or at least 15 g/L, or at least 16 g/L, or at least 17 g/L or at least 18 g/L, or at least 19 g/L, or at least 20 g/L, or ranging at about 3 g/L to about 18 g/L, or about 3 g/L to about 15 g/L, or about 3 g/L to about 12 g/L, or about 6 g/L to about 9 g/L, or about 7 g/L to about 8 g/L. The liquid form of the extract composition may also be measured in millilitres, with 1 gram equivalent to 1 millilitre.

It will be understood that the amounts of the composition added may be adjusted as desired. For example, the amounts used may be varied depending on the other ingredients being utilised (e.g., yeast strains, bacterial strains, enzymes, hops or hops component(s), etc), or depending on whether the composition has been formulated to include one or more additional ingredients (e.g., one or more yeast strains, bacterial strains, enzymes, hops or hops component(s), etc). Amounts added may also be adjusted depending on the concentration of thiol precursors in the composition.

When preparing fermented beverages, it is expected that addition of the composition prior to fermentation will increase thiol precursors levels as compared to pre-fermentation mixtures prepared without the composition. For example, the levels of C3MH may increase 2 fold to 9 fold, or at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, or at least 20 fold. The levels of CG3MH may increase 1.05 fold to 1.2 fold, or at least 1.05 fold, at least 1.1 fold, at least 1.5 fold, or at least 2 fold. The levels of GC3MH may increase 1.1 fold to 1.4 fold, or at least 1.05 fold, at least 1.1 fold, at least 1.2 fold, at least 1.3 fold, at least 1.4 fold, at least 1.5 fold, at least 2 fold, or at least 3 fold. The levels of G3MH may increase 1.2 fold to 1.8 fold, or at least 1.2 fold, at least 1.3 fold, at least 1.4 fold, at least 1.5 fold, at least 1.6 fold, at least 1.7 fold, at least 1.8 fold, at least 2 fold, or at least 3 fold. In particular, when preparing brewed beverages, such increases in thiol precursors may be seen in the wort.

Therefore, with the grape skin extract provided as part of a beneficial composition (e.g., powder, flakes, etc), this provides an ideal ingredient for use in the fermentation process. In particular, the composition of the present disclosure provides a stable source of volatile thiols and their precursors, which can be introduced into the fermentation process to increase highly desirable aromatic compounds (e.g., tropical, fruity, etc) in a finished beverage. The production of beers, beer-wine hybrids, fruit beers, and ciders is specifically envisioned, along with production of kombuchas. For example, to brew highly aromatic beer, it is possible to use the grape skin extract composition as a replacement for hops, i.e., fermentation without the addition of hops. Alternatively, at least one hops in combination with at least one grape skin extract composition may be introduced to the active fermentation. In various alternatives, the hops may be combined with the grape skin extract composition before or upon introduction to fermentation mixture, and the hops may be provided in whole leaf form or as hops component(s), e.g., hops powder, hops extract (e.g., lupulin, hops terpenes, and/or hops esters), hops pellet, or hops plug. In one particular aspect, the introduction may be carried out as part of a dry hops addition. Notably, the combination of hops and grape skin extract being introduced into the active fermentation allows for the biotransformation to produce of aromas and flavours.

Accordingly, in various aspects, this disclosure encompasses combinations which include the composition comprising grape skin extract and at least one additional ingredient. This includes, for example, one or more hops, one or more hops component(s) (e.g., lupulin, hops terpenes, and/or hops esters), one or more yeast, one or more enzymes, one or more fruit concentrates (e.g., juice, flesh, or whole fruit, or combinations of these), one or more sulphur compounds (e.g., any thiol precursor or combinations of these), or other ingredients as set out herein, or any combination of the preceding. In certain embodiments, the grape skin extract composition may be provided in liquid or semi-liquid form. That is, the composition may be mixed in water or other solvent(s), including any aqueous solution as described herein. It is possible to combine the grape skin extract composition of this disclosure with one or more other grape skin concentrates, for example, grape skin concentrates prepared from dried skins (see, e.g., PCT/NZ2021/050090). In certain embodiments, the grape skin extract composition may be provided as a dry form (e.g., powder). In further embodiments, the one or more additional ingredients may be provided in liquid, semi-liquid (e.g., paste, gel), or dry (e.g., powder) form to produce the combination.

Particularly encompassed are combinations comprising the grape skin extract composition and at least one yeast. As an example, the grape skin extract composition may be combined with one or more yeast strains used for volatile thiol production, as described in detail herein. Also encompassed are combinations comprising the grape skin extract composition and one or more hops and/or hops component(s). As an example, the grape skin extract composition may be combined with one or more hops/hops component(s) used for volatile thiol production, as described in detail herein. Further encompassed are combinations comprising the grape skin extract composition and one or more hops or hops component(s) and also one or more yeast. For example, the grape skin extract composition may be combined with one or more hops or hops component(s) used for volatile thiol production and also one or more yeast used for volatile thiol production. Other combinations may be obtained and used in the methods set out herein.

It is envisioned that the composition of this disclosure may be included in kit, for example, a kit for preparing a beverage, such as a fermented beverage. The composition may be provided in one or more containers in the kit. Additional components may also be provided with the kit, for example, one or more hops or hops component(s), or one or more yeast, one or more bacteria, one or more enzymes, one or more sulphur compounds, one or more fruit concentrates, or any combination thereof. The composition may be provided as a combined formulation as described in detail herein. Optionally, instructions may be provided with the kit, as well as any other item, such as any number of containers, labels, and/or tools, including bottles, injectors, scoops, etc. The instructions for the kit may include information as to storage, usage, or testing, amongst other information. The kit may optionally include one or more reagents for testing thiol precursor levels or volatile thiol levels.

### EXAMPLES

The examples described herein are provided for the purpose of illustrating specific embodiments and are not intended to limit this disclosure in any way.

### Example 1: Processing of Grapes

High thiol content grapes were sourced from Nautilus Estate in Marlborough, New Zealand. Predominately Sauvignon Blanc MS clone (UCD Clone 1) were used, alongside Bordeaux clones (Clone 316 and 317). Blocks noted for their historically high thiol content were selected, and harvested using a Gregoire and Pellenc machine destemming harvesters. The grapes were harvested between 21.5 and 22 degrees Brix. During the harvesting, potassium metabisulphite was dosed at 66 grams per tonne and added to the destemmed grapes. The grapes were put through a crushing machine with 30 parts per tonne ascorbic acid and 20 ml per tonne of enzymes added (pectolytic enzymes). The crushed grape must was then transferred through a must chiller set to 12° C. and into a holding tank for 1 to 8 hours.

A 150 HL Diemme open screen press was used to extract and separate the juice from the skins. The press operated at a maximum pressure of 2 bar over a 150 to 180 minute long press cycle. The press operated at an ambient temperature with the grapes added at 12° C. and remaining at a similar temperature throughout pressing. Separate lots of skins were carefully collected and frozen immediately post press. Pressing was performed at 720 litres per tonne, or 750 litres per tonne.

The skins were collected immediately from the press into plastic lined bins and given an additional dose of potassium metabisulphite and ascorbic acid (same dosages of each, as noted above). The bins of skins were kept in a cold room at 0° C. until collected in a refrigerated truck and transferred to a freezer facility where by they were frozen at −20° C. The frozen skins were thawed gently over a 24 hour to 72 hour period at ambient temperatures, until in a free form, pliable state.

### Example 1A: Extraction Process

In one extraction process, thawed grape skins were added to a volume of pure water between 2 to 6 times the volume of grape skins. The mixture was stirred at room temperature for 1 hour and left to settle for 10 minutes. After that, the liquid was filtered from the solids by use of a brewing filter. The liquid extract was then dried down in vacuo. The resulting product resembled a dark green paste. The paste was then freeze dried and milled into a powder as in Example 1. The concentrations of thiol precursors in the powder from this method were: Cys-3SH—1.19 mg/kg and GSH-3SH—3.76 mg/kg.

### Example 2: Production Method Parameters and Analysis

### Overview

These methods look to develop grape skin extraction methods. One feature of an extract compared to a whole product is reduced sedimentation in the final beverage.

Our earlier studies found that water extraction at 5° C. was not an effective extraction method, even after seven days. Extraction in water at 20° C. extracted more thiols compared to 13° C.

The current experiments were proposed to establish key conditions for a liquid extraction of grape skins. The tested parameters included temperature, extraction time, and the effect of leaving the seed in the extraction mixture.

### Experimental Method

The grape skins were pressed, frozen, and thawed as in Example 1. The starting material was a mixture of skins and seeds. For one portion of the starting material, seeds were manually removed to make a ‘skin only’ material. The other portion retained the seeds. The wet skins made up 63.06% of the wet as-received material.

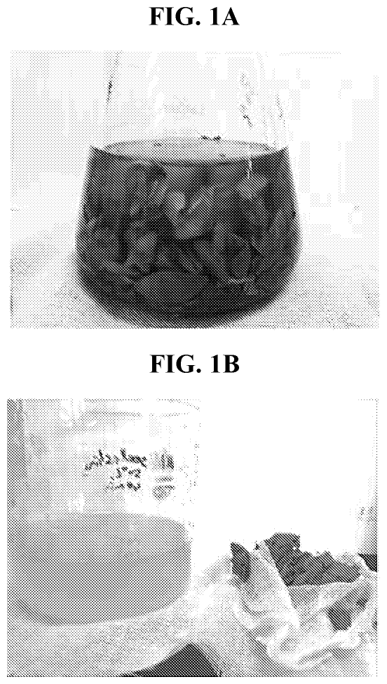

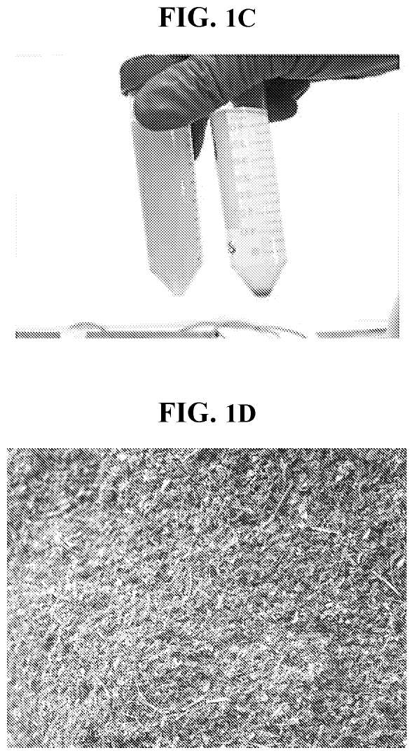

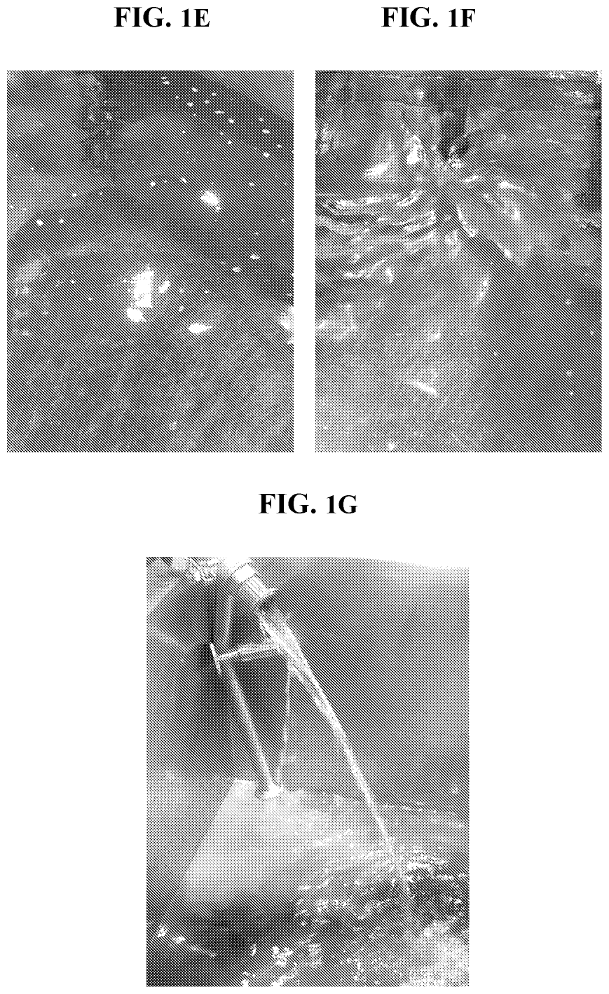

Approximately 30 g of the wet skin (or skin+seed) was measured into shake flasks with 150 g of RO (reverse osmosis) water, giving a skin to solvent ratio of 1:5 (w/w) (FIG. 1A). The flasks were placed in a heated shaker at 30, 50, and 70° C. At time intervals of 40, 80, and 120 minutes, a single flask was removed and pressed through a muslin cloth. (FIG. 1B).

The filtrate was centrifuged at 2850 RCF for 10 minutes to remove the larger particulates, then filtered through 11 m paper to polish the centrate. The filtrate was analysed for small molecules. (FIG. 1C). Ascorbic acid was added (0.01% w/w) to a selection of the trial conditions. Ascorbic acid is generally used to retard oxidation and enzymatic browning.

### Extraction Results

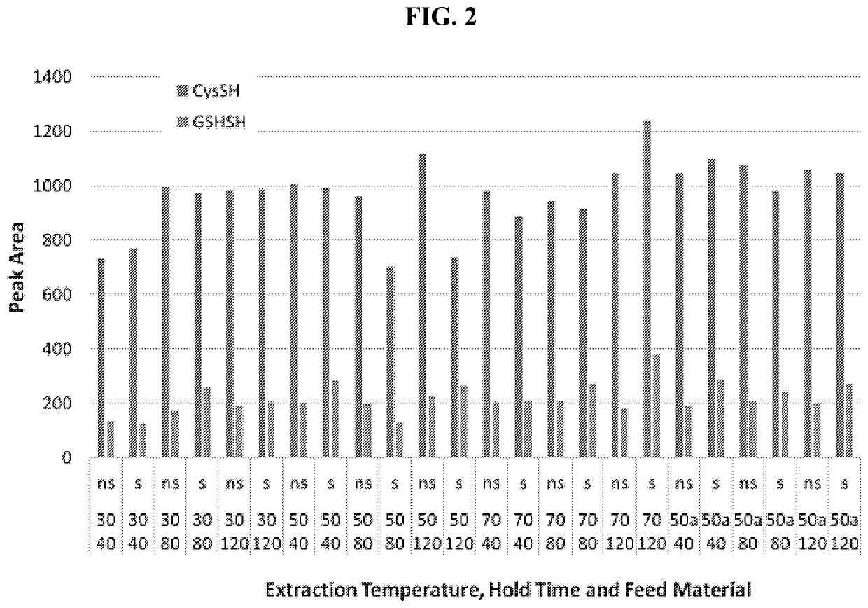

Thiol precursors: The extraction and separation of the aqueous phases were carried out with minimal complications. There were no processing issues of note. The effect of the extraction temperature and time varied with molecule class. For the target thiol precursors, the highest levels were observed at 70° C. and 120 minute incubation (FIG. 2). However, the differences were not statistically significant when comparing the data from lower times and temperatures. In general, there was higher GSH-3SH levels with the material that contains seeds compared to non-seeds, but this was less than one standard deviation.

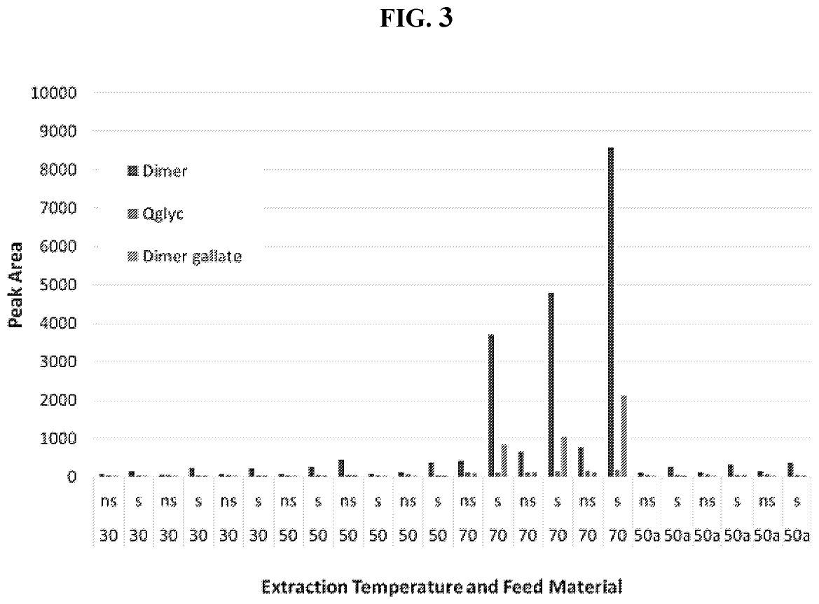

Polyphenols: The recovery of polyphenols was relatively low at 30° C. and 50° C., with the concentration. increasing with temperature and extraction time (FIG. 3). The catindimer (dimer) is the primary polyphenol extracted, but the trend for increased extraction of polyphenols occurred for the quercetin-glycosides (Qglyc) and catindimer gallate (dimer gallate). Where polyphenols are not desired, it is therefore possible to use higher extraction temperatures (e.g., greater than or equal to 50° C.) and/or reducing the levels of seeds (e.g., removing seeds).

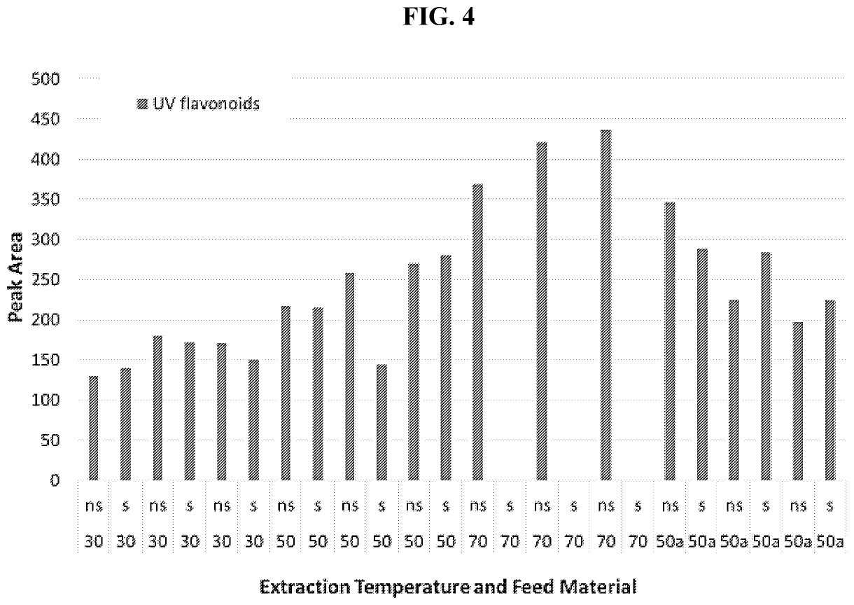

Flavonoids: Flavonoid levels also showed a clear relationship between temperature and extraction time (FIG. 4). In this case, there is little difference between the inclusion and exclusion of the seeds. Therefore, the flavonoids extraction results might be the key parameter for the final extraction conditions. For example, if the flavonoids are wanted in the final product, extracting with a higher temperature (e.g., greater than or equal to 50° C.), a long hold period (e.g., greater than 50 minutes), and the reduction of seeds (e.g., seed removal) will be useful. On the other hand, if they are not desired, extraction can be performed at a lower temperature (e.g., 30° C. or less) and a moderate extract time (e.g., about 30 to about 50 minutes).

### Conclusions

The key conditions depend on the three small molecular classes being extracted. The precursor thiols were extracted well at mid- to high temperatures, 30° C., 50° C., and 70° C. The polyphenolics were extracted best at the higher temperature (70° C.), particularly with seeds present. Flavonoids were extracted best at higher temperature (50° C. and above), but not significantly affected by the presences of the seeds. Larger scale studies were proposed to include incubation at 50° C. for 45 minutes.

### Example 4: Scaled Up Production and Analysis

### Overview

The main target of these experiments were thiol precursors. In wine, thiol precursors increase the flavour in aromatic wines. These experiments were proposed as a larger-scale extraction of grape skin. In addition, methods were employed to produce powders and other dried products.

### Experimental Method

The grape skins were pressed, frozen, and thawed as in Example 1. The starting material was a mixture of skins and seeds. The grape skin with seeds was extracted using 50° C. water.

A triple extraction was used to increase the solids loading of the filtrate to increase the mass of product recovered. This means that the filtrate from the first extraction was reused, extracting a second round of grape skins, and this was repeated with a third round of skins.

For each of the three extractions, 120 L of water was mixed with ˜30 kg of skin for 45 minutes, then the material was pressed using a hydropress at a maximum of 1 bar. The filtrate out was 122 kg, i.e., slightly higher than the starting water mass. In total, 96.26 kg of grape skin/seed was extracted.

The filtrate was clarified using a Koch 6″ hollow-fibre wine filter with a pore size of 0.3 m. The system was operated for 3 minutes, with a transmembrane pressure of 0.94 bar. The surface area was 9.8 m². No diafiltration was used, 125.3 kg of permeate was recovered, and 8.56 kg of retentate remained. Both the permeate and retentate were freeze dried.

### Extraction Results

Analysis showed that the solids content of the permeate was 7.25% (w/w). The retentate solids were 9.68% (w/w) (Table 5). There was a reduction in the yield when comparing the difference between the 1^{st}/2^{nd} extractions and the difference between the 2^{nd}/3^{rd} extractions (Table 5). Therefore, in certain circumstances, a double extraction may be utilised rather than a triple extraction. This will decrease the water and processing volume.

TABLE 5
Thiol precursors in filtrate, permeate, and retentate fractions

| | Total Solids |
|---|---|
| | (g/100 g] |
| Filtrate #1 | 3.25 |
| Filtrate #2 | 6.02 |
| Flitrate #3 | 7.41 |
| Permeate | 7.25 |
| Retentate | 9.68 |

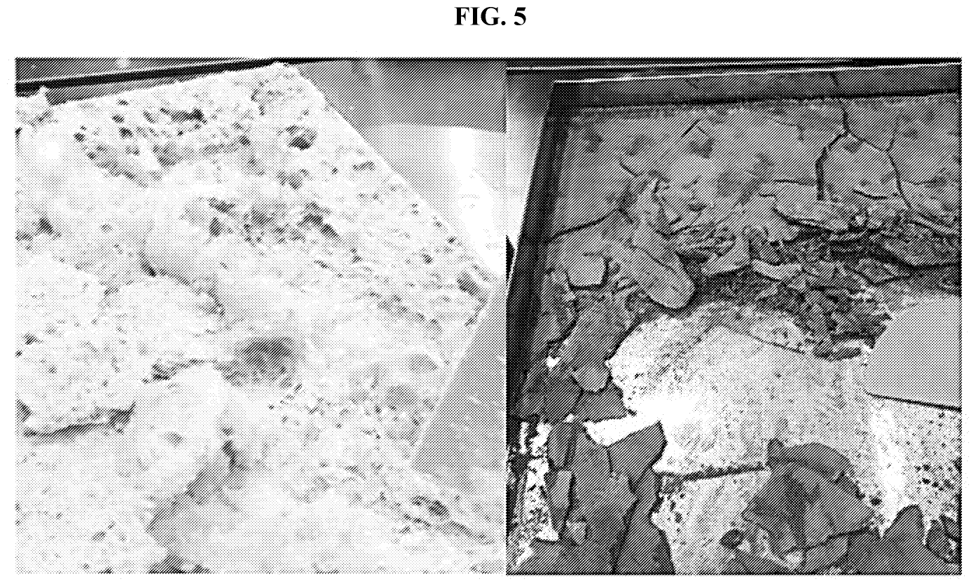

Both the permeate and retentate were freeze dried. The permeate foamed (FIG. 5A). The retentate material formed a skin as it was freeze dried, but in general freeze dried well (FIG. 5B). The permeate was also evaporated under vacuum inside a Buchi bowl. This formed a concentrated paste.

There was a difference noted in Cys-3SH and GSH-3SH concentrations (Table 6). The evaporated material had a lower concentration. This may be due to the time required for the evaporation process. Therefore, faster drying times may be more favourable.

TABLE 6
Thiol precursors in permeate and retentate fractions

| | CycSH | GSHSH |
|---|---|---|
| | [Peak | [Peak |
| | Aren/100 mg dry] | Area/100 mg dry] |
| MF Permeate F/D | 71.2 | 20.3 |
| MF Permeate Evaporated | 51.6 | 12.5 |
| MF Retentate F/D | 48.9 | 9.2 |

Further results are shown in Table 7, below. Shown are the precursor thiol levels for the concentrated liquid extract composition and the freeze dried powder produced from the liquid extract composition. The results demonstrate that the different preparative methods produced compositions with similar levels for each of the precursor compounds tested (Table 7). We have observed that certain batches of grapes produce compositions with higher levels of thiol precursors. This can be attributed to the time of harvest, and the historical levels of thiols found in the individual blocks of grapes. Accordingly, it may be useful to estimate thiol precursor levels in the grapes in advance of processing, and target specific growing blocks and/or harvest windows.

### Conclusions

These studies showed that when processing in a batch-wise process, there is the potential for a grape skin filtrate to be reused to increase the concentration in the filtrate. A continuous counter current extraction process may be particularly useful.

In view of the results from Example 3, there are different options available for the extraction parameters. Where flavonoids are desired, then a higher extraction temperature (e.g., 50° C. or above, or 70° C. or above) may be used, if not then lower temperatures (e.g., about 30° C. to about 50° C.) may be used. Where higher temperatures are used, and where phenolics are not desired, then seed reduction (e.g., seed removal) may be useful.

In addition, there is a potential for using reverse osmosis protocols to obtain the grape skin extract compositions.

### Example 5: Double Extraction Process and Analysis

Extraction 1 (50° C.): The grape skins were pressed, frozen, and thawed as in Example 1. The vessel was half loaded with hot water (50° C.), then the skins (200 to 250 kg) were loaded into the extraction vessel. The final skins to water ratio was 1:4 (w/w). The extraction time of 80 minutes was used, with the skins removed using a decanter (FIG. 1D). The supernatant was loaded back into an extraction vessel for another load of skin.

Extraction 2 (70° C.): Performed similar to Extraction 1, but the second extraction was carried out at 70° C. The final solutions were collected and frozen in 2 L, 20 L, or 200 L drums.

After the second extraction, the skins were removed via decanter, then the solution was clarified using microfiltration (Pall® hollow fibre system). The microfiltration permeate was cooled, followed by concentration using a reverse osmosis membrane (Dow Filmtec RO-3838/30-FF; FIGS. 1E-1G). The potential maximum operation pressure for reverse osmosis was 40 Bar.

After concentration, the solution was chilled, and ideally frozen to stop any biological activity. Analytical samples of the decanter supernatant, microfiltration permeate, reverse osmosis concentrate was collected and frozen. Inline samples throughout the process were sampled for brix. The resulting grape skin extract concentrate was then freeze dried into a powder. The powder and the liquid extract samples were analysed for thiol precursors.

The results are shown below. As before, the different preparative methods produced compositions with comparable thiol precursor levels.

| | Cys-3SH/g solid (μg/g) |
|---|---|
| Freeze dried reverse osmosis concentrated extract | 1.811781684 |
| Freeze dried microfiltration extract | 1.94149326 |
| Reverse osmosis concentrated liquid extract | 1.502372735 |
| Microfiltration liquid extract | 1.732528055 |

### Example 6: Beverage Preparation Methods Using Thiol Boosting Yeast

100% barley malt fermentations were used with a target original gravity of 15° P. The extract composition from Example 4 was added to the whirlpool when temperature was >180° F. For the liquid form of the extract composition, dosage was 6 g/L. For the powder form of the extract composition, dosage was 3 g/L. Rest was for 15 minutes. As a control, parallel experiments were performed in the absence of the grape skin extract composition. The wort was transferred and cooled to 70° F. Cosmic Punch™ yeast (Omega Yeast; OYL 402) was added at 10 million cells/ml/° P (15 million cells/ml). Fermentation volumes were 300 ml. Fermentation was anaerobic with air locks at 70-72° F. After 14 days of fermentation, samples were transferred off of the yeast slurry to assess. Samples were decanted and stored at −80° C. in 50 ml conical tubes with the addition of sodium metabisulfite, to assist in preventing oxidation of thiols. In parallel, 50 ml was frozen with 15 ppm sodium metabisulfite. Where wort precursor levels were measured, samples were frozen pre-fermentation after the whirlpool step. Samples were sent for UPLC-MS/MS analysis, using a stable isotope dilution assay (Nyseos Laboratories, France).

Results are shown in Table 8, below. This table shows volatile thiol levels for beer samples: 40-2 (control), 40-16 (prepared with concentrated liquid extract composition), and 40-18 (prepared with freeze dried powder form obtained from liquid extract). The table also shows precursor thiol levels for wort samples: 40 (control wort), 40 Ex-L (prepared with concentrated liquid extract composition), and 40 Ex-D (prepared with freeze dried powder form obtained from liquid extract) The results demonstrate that the grape skin extract compositions produce increases in precursor thiol compounds in the wort, including significantly increased levels of C3MH, and increases also in GC3MH and G3MH (Table 8). The grape skin compositions further produce significant increases in certain volatile thiol compounds in the resulting beer, including increased levels of 3MH (Table 8). It is believed that higher levels of thiol precursors may be achieved for the composition with shorter periods (or omission) of evaporation under vacuum. This in turn can be used to achieve higher levels of volatile thiols in the products (e.g., beverages) obtained with the composition.

### Example 7: Further Production and Processing Methods

### Overview and Summary

The objective of this work was to process several batches of grape skin with seed through a commercial process and to generate bulk fractions. One advantage of liquid extract compositions is the reduced levels of sediment in the final beverage.

These experiments were carried out to utilise commercial scale extractions for the grape skins and seeds and to utilise reverse osmosis operation on the clarified solution to concentrate the precursor compounds.

Two processing conditions included methods utilising: (1) extraction at 50° C., which had the highest thiol precursor extraction rates, alongside the lowest polyphenolic extractions rates; and (2) extraction at 70° C., which in addition to high thiol precursor extraction rates, appeared to have good biological control, and polyphenolics which could potentially improve the shelf-life.

### Extraction

The grape skins were pressed, frozen, and thawed as in Example 1. The as-received material was a mixture of skins and seeds. The top layer of skin was removed due to the presence of mould on the surface. The extraction/mixing tank was filled with hot water. This was done so that water was already in the vessel before loading. Next, 250 kg of skin was weighed and loaded into each of the extraction vessels via a screw conveyor.

The mixture was circulated through a heat exchanger to keep the material at 50° C. (or 70° C.). After −80 minutes, the material was fed into a decanter out from the bottom outlet. The skin, pulp and seed were removed. The decanter removed more water from the skin and pulp than what was in the feed material. This showed that there was the potential for directly running the skins through a screwpress or decanter to reduce the amount of water for freeze drying.

The liquid was drained into a collection bin and pumped past a flow meter (measuring the amount of supernatant) heat exchanger. The final solids from the bottom of the decanter were weighed. The solids from the final run R4/1 were mixed with washing water, so the mass was excluded. The solids of the supernatant were measured using a refractometer.

The mass of the skins in and out, plus supernatant, is shown in Table 9, below. Regarding the sample numbering: R1 and R2 were extracted at 50° C. R3 and R4 were extracted at 70° C. R1, R2, R3 were microfiltered and RO processed as described further below. R4 was only microfiltered. R1/i, R2/1, R3/1 indicate single extractions, R1/2, R2/2, R3/2 indicate double extractions. Double extractions are described previously.

TABLE 9
Masses for grape skin extraction samples

| Extraction | Solids in | Solids out | Supernatant | Brix |
|---|---|---|---|---|
| Extraction R1/1 | 250 | 112 | 1094 | 3.0 |
| Extraction R1/2 | 250 | 132 | 1224 | 5.9 |
| Extraction R2/1 | 250 | 154 | 1162 | 3.4 |
| Extraction R2/2 | 250 | 158 | 1378 | 5.7 |
| Extraction R3/1 | 250 | 127 | 1103 | 2.6 |
| Extraction R3/2 | 250 | 131 | 1240 | 6.2 |
| Extraction R4/1 | 250 | — | 1182 | 3.8 |

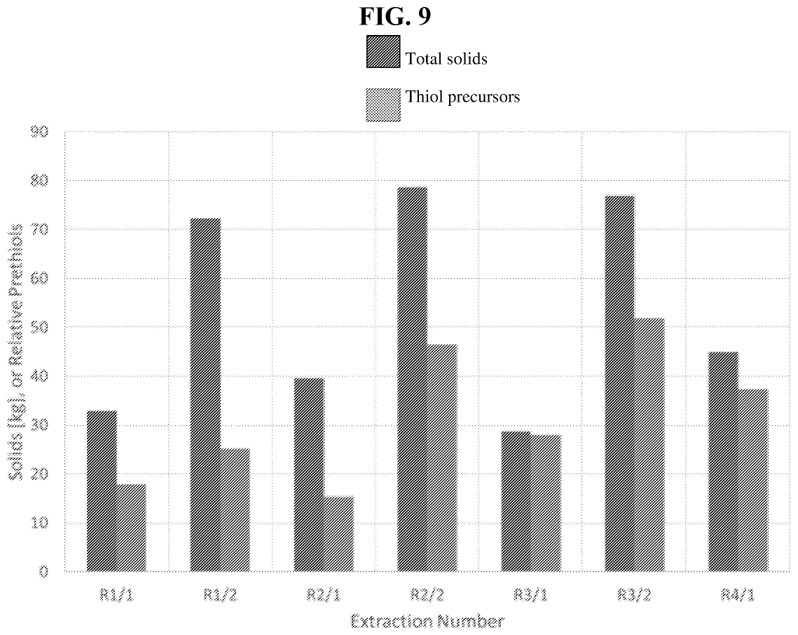

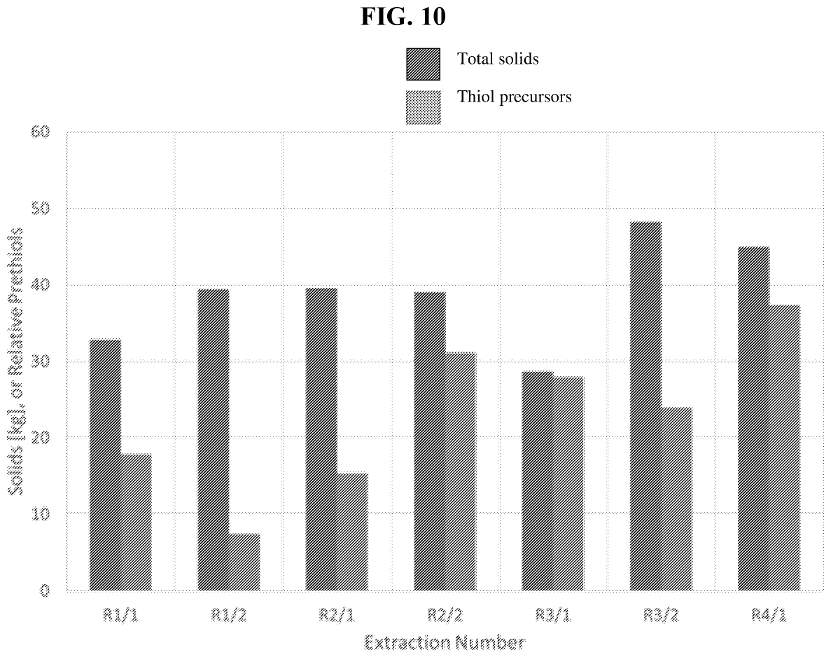

Table 9 shows the volume of extract and brix obtained at 50° C. (R1 and R2), and 70° C. (R2 and R3) and with double extractions (R1/2, R2/2, R3/2). Further analysis for these samples is provided in FIGS. 9 and 10.

FIG. 9 shows the total solids (left hand bar for each pair of bars) recovered from the supernatant for each sample and the levels of thiol precursors in the supernatant for each sample (Cys-3SH and GSH-3SH). This figure sets out the sample analysis.

FIG. 10 shows the total solids (left hand bar for each pair of bars) recovered from each sample and the levels of thiol precursors from each sample (Cys-3SH and GSH-3SH). This figure sets out the calculated solids levels for each batch.

The total solids in the supernatant was calculated from the brix and the mass. In general, the mass of the solids in the supernatant after the second extraction increased by a factor of 2. This is expected, and the results parallel those obtained for the smaller scale work. The levels for thiol precursors, 3-mercapto-1-hexanol (Cys-3SH) and 4-methyl-4-mercapto-2-pentanone (GSH-3SH) were also determined. The higher temperature R3 had the highest overall levels of thiol precursors, and the final extraction R4 had the highest overall amount of thiol precursors in a single steep extraction, i.e., higher levels than R1/i, R2/1, R3/1 single extractions. The R2/2 double extraction did obtain more thiol precursors than the R3 extraction

The results show that the higher extraction temperature does appear to extract more of the thiol precursors. The results were similar to those observed for the smaller scale preparation methods. The effect of the double extraction on the thiol precursors is shown in FIG. 10 (right hand bars). The increase in the thiol precursors for double extractions was 41% for the R1 extractions, and 303% for the R2/2, and for the R3/2 set at 70° C. had an 85% increase compared to a single extraction.

Further analysis for determining the concentration of polyphenols is shown in Table 11. Regarding the sample numbering, MF50 refers to 50° C. extraction with microfiltration for samples R1 and R2. MF70 refers to 70° C. extraction with microfiltration for samples R3 and R4. R050 refers to 50° C. extraction with reverse osmosis for R1 and R2. R070 refers to 70° C. extraction with reverse osmosis for R3. FD refers to freeze dried samples. The microfiltration, reverse osmosis, and freeze drying methods are described further below.

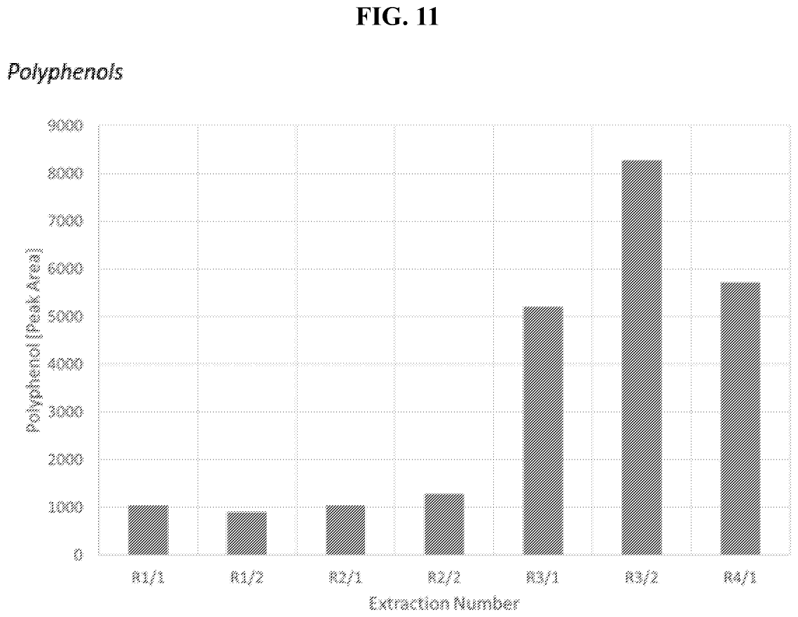

The results show that the recovery of polyphenols was relatively low at 50° C. and quite high at 70° C. (FIG. 11). Without wishing to be bound by theory, it is believed that the polyphenols are being extracted from the seed remaining in the skin, and that the higher the temperature, the better for extraction for polyphenol.

### Microfiltration

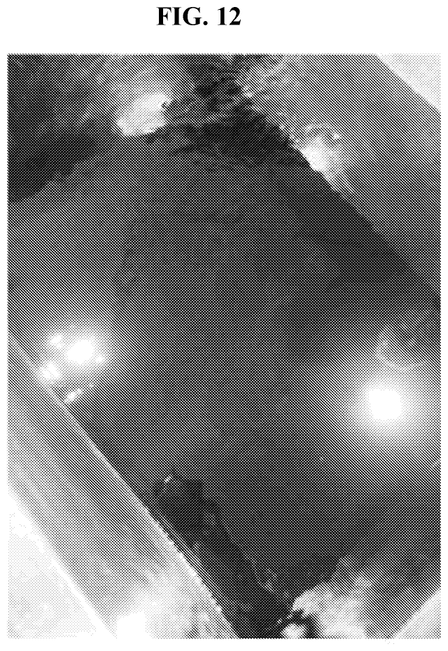

The supernatural after the second extraction was clarified through a microfiltration unit, using Pall's hollow fibre membranes. Pall hollow fibres are made from PVDF, with a nominal pore size of 0.1 μm. The unit has a significant hold-up volume of approximately 500 L. This results in a loss of permeate from the first run in the morning. Processing wise, this was a quick operation due to the large surface area of the unit. The solution was very clear, as shown in FIG. 12.

### Reverse Osmosis

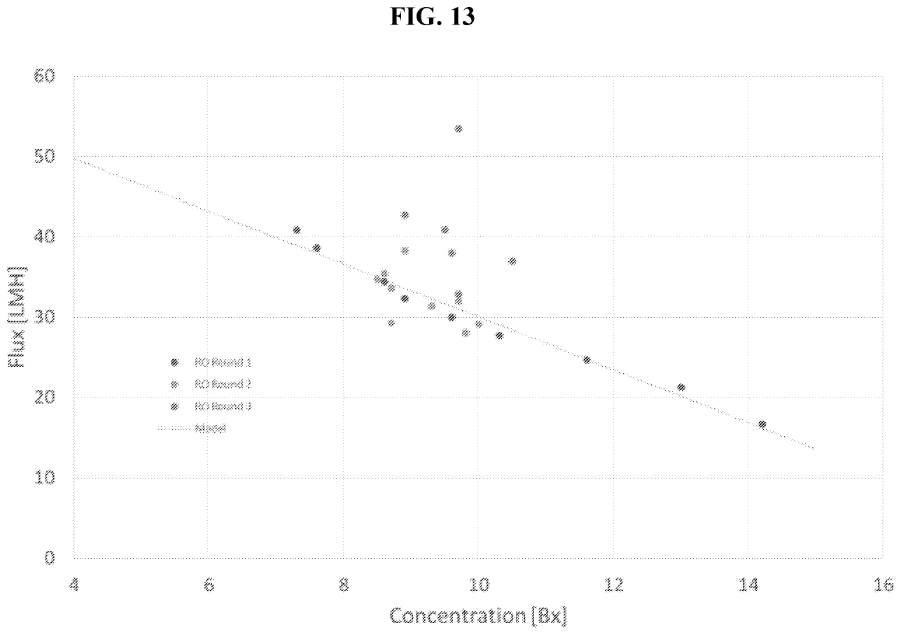

Reverse osmosis was used to increase the concentration of the thiol precursors. A DOW Flimtec 3838 (7.5 m²) membrane was utilised. In brief, the results showed that the process worked in terms of retaining the thiol precursors and removing the water. The flux rates are shown in FIG. 13, which show the decrease in the flux rate as the concentration of the solution increase due to the osmotic pressure on the solution. The system was made up of an eSV1 vertical pump to supply the booster pump (Danfoss APP0.8 piston). This unit provided the pressure.

The retentate was circulated with a pressure drop on 0.8 bar over the membrane, with an estimated flow rate of 7 m³/hr. The maximum operating pressure is 40 barg, limited by the pressure rating on the head of the circulation pump. The pressure was controlled by a needle valve, which regulated the flow of material back to the feed tank. The APP 0.8 has a maximum flow rate of 1000 L/hr. Ideally, the pump will run at the maximum flow rate, with the permeate flow rate plus tank return flow rate equal to 1000 L/hr. As the permeate rate drops, the return valve is increased to keep the processing pressure the same.

In the initial run, (R1), using the microfiltration permeate as the feed material. The feed tank was filled (FIG. 12), then operated in a batch system. That is, no new material was added, and the solution was concentrated, with the retentate (concentrate) returning to the feed vessel. The solution concentrated well until the protection filter (on the feed tank outlet) was blocked. After the filter blockage, the 140 L of concentration was collected into jerrycans. By that stage, 301 L was removed from the system, sent to drain. The permeate was clear and colourless, with a brix of 0.0. See Table 10. No measurable thiol precursors were lost through the membrane.

In the second run (R2), as the solution has a critical limitation to concentration, the microfiltration permeate was added to the vessel, and the RO plant concentrated as much material as possible. 739 L of permeate was discarded, and 1070 L was packed out. As soon as the retentate returning to the tank was above ten brix, the retentate line was fed into the 200-L bins and jerrycans, with new material added to the feed tank.

The third run (R3) was operated as a batchwise system. 190 L of concentrate was packed out, with 209 L of permeate sent to drain. The tartrates, which are present in grape extraction/juices, reached a critical concentration around 14.2 brix. In some circumstances, precipitation was seen after approximately half the water being removed, i.e., around 14% (w/w). The results confirmed that the target compounds are being retained by the membrane. See Table 10, below.

TABLE 10
Reverse osmosis analysis (direct infusion)

| Sample | pH | Brix | Cys-3SH | GSH-3SH |
|---|---|---|---|---|
| RO Permeate | 4.28 | 0 | 0 | 0 |

The flux curve was calculated using the following formula:

$$J [ LMH ] = \frac{A}{4.537} ( Δ {P}^{TMP} - 1.986 C )$$

Where j is the flux rate, in litre, per meter squared of membrane area per hour, A is the surface area of the membrane in m², ΔPTMP is the transmembrane pressure in bar, and C is the concentration of the retentate in brix.

From the results obtained, there was no evidence of the thiol precursors passing through the reverse osmosis membrane or being damaged by the reverse osmosis process. A top concentration of approximately 14 g/100 g was observed for the system utilised. For this system, the osmotic pressure was approximately 2 bar per 1% solids of the clarified solution.

### Freeze Drying

The effect of freezing drying on the thiol precursors was compared by analysing the sample as received and a sample after freeze drying. The test was done on two samples; the microfiltration permeates and reverses osmosis concentration from the first extraction run. The results are included in Table 11, below. In brief, the results showed that the freeze drying process does not damage the thiol precursors.

### Observations and Conclusions

It was noted that, in some cases, the marc adhered to the outside of the decanter shell. Methods for dislodging the marc, other than rinsing the decanter shell, may be helpful for streamlining the process. Where microfiltration units are utilised, the solution may be cooled before the filtration process. Heat exchange systems may be used to achieve this, and the heat may be transferred to the earlier extraction steps, e.g., 50° C. or 70° C. incubations. Where there is additional fluid in the system, this may be used to offset the hold up volume for the microfiltration unit. If more fluid is used with the same mass of compounds, this means that more compounds can be collected.

Single steep extractions, i.e., decoupling of the two extraction tanks, may also be useful. For example, depending on whether yield or throughput are more important, the product can be single or double extracted. Double extraction enables greater material throughput and requires less water removal but does cost a small amount of yield.

In addition, it may be useful to utilise a continuous counter-current extraction process. Where the solution is more dilute, this may be readily concentrated in a reverse osmosis system to below the critical limit, meaning that the water can be recycled. In such circumstances, more grape skin can be processed in the same amount of time.

The extraction and solids separation may be performed by methods similar to those used herein. For example, a single 34.4 m² 8″ membrane at 20 LHM (at 13 brix) has a processing rate of 688 L per hour. Accordingly, it should be possible to utilise larger reverse osmosis membranes (e.g., at least 8″) to concentration the solution, thereby increasing the flow rate moving through the system.

For minimising concentration and/or drying costs, one focus will be on the ability to concentrate the thiol precursors in the aqueous solution. One option is to utilise cation ion exchange for removing the tartrate ions from solution, then concentrating the solution with reverse osmosis. For storing the final composition, 200 L seal drums may be utilised, or fully sealed bladders.

Whether polyphenols are desired will determine the selected extraction temperature. If flavonoids are desired, then a high extraction temperature (e.g., 70° C.) may be utilised; if flavonoids are not desired, then a cooler extraction temperature may be utilised (e.g., 50° C.). The results also showed that reverse osmosis is suitable for the concentration of the thiol precursor compounds and there was no damage/loss of the thiol precursor compounds from freeze drying.

### Example 8: Beverage Preparation Using Thiol Boosting Yeast

The liquid extracts R1, R2, R3, R4 were obtained as described in Example 7. The double extractions for each of R1-R3 were utilised in these experiments (i.e., R1/2, R2/2, R3/2; see Example 7). Beverage preparation methods were employed as described in Example 6, except where noted.

The control utilised yeast strain OYL-402 with no liquid extract additions. For the test samples, each liquid extract sample was added to separate vats of hot wort. A standardised addition rate of 6 g/L or 12 g/L was used. This was calculated as shown in Table 12-1, below.

TABLE 12-1
Calculations for liquid extract additions

| | | 6 g/L equivalent | 12 g/L equivalent |
|---|---|---|---|
| | Total solids | addition | addition |
| R1 | 13% | 46.40 ml | 92.81 ml |
| R2 | 9% | 68.42 ml | 136.83 ml |
| R3 | 11% | 56.07 ml | 112.15 ml |
| R4 | 4% | 150.38 ml | 300.75 ml |

The results are shown in Tables 12-2, 13-1, 13-2, below. Beer produced using the extract compositions showed significantly higher levels of 3MH and very positive sensory data, including increased beverage clarity and reduced sulphur taste/smell compared to the controls.

In particular, the liquid extracts were observed to impart a pleasant and pure tropical aroma, with relatively low levels of colloidal haze. Without wishing to be bound by theory, it was proposed that higher polyphenol levels could contribute to haze formation. In this way, it could be possible to employ the disclosed liquid extracts to either avoid or promote haze development, if polyphenols were omitted or added, respectively.

### Example 9: Beverage Preparation Using Thiol Boosting Yeast

Brewing trials were performed to assess and compare the results produced by OYL-011, OYL-402, or OYL-405 yeast strains (Omega Yeast), and the inclusion of extract R3, or inclusion of no extract (controls). The liquid extract R3 was obtained as described in Example 7. The double extraction sample for R3 was utilised in these experiments (i.e., R3/2; see Example 7). The beverage preparation methods were employed as described in Example 6, except where noted.

All yeast were propagated in flasks for 2 days prior to brewing. Brewhouse wort method was utilised. The dosage rate of the R3 extract was 4.3 g/L in the whirlpool stage. Control 1 included OYL-11 yeast with no extract addition. Control 2 included OYL-402 yeast with no extract addition. Control 3 included OYL-405 yeast with no extract addition. Each pitcher except for the controls had 12.9 g of extract added prior to filling with sterilized wort (3 litres). After a short secondary rest of 10 minutes, the wort was siphoned into another sanitized pitcher, and decanted into flasks. Each flask had 300 mL of wort added, was chilled, inoculated with 2 mL of slurry, aerated for 20 minutes, and capped with a sanitized airlock. Fermentation was carried out for 2 weeks. Samples were taken from each flask and sensory data was obtained.

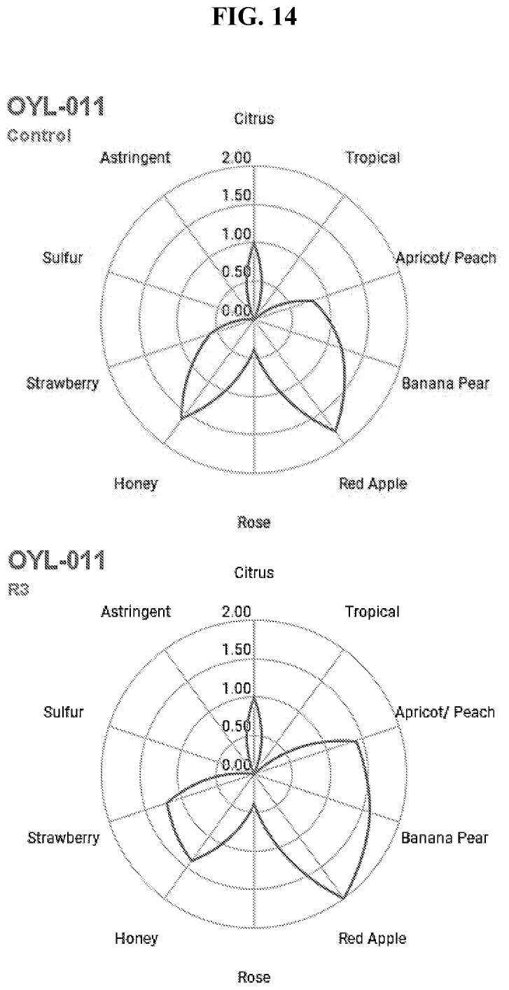

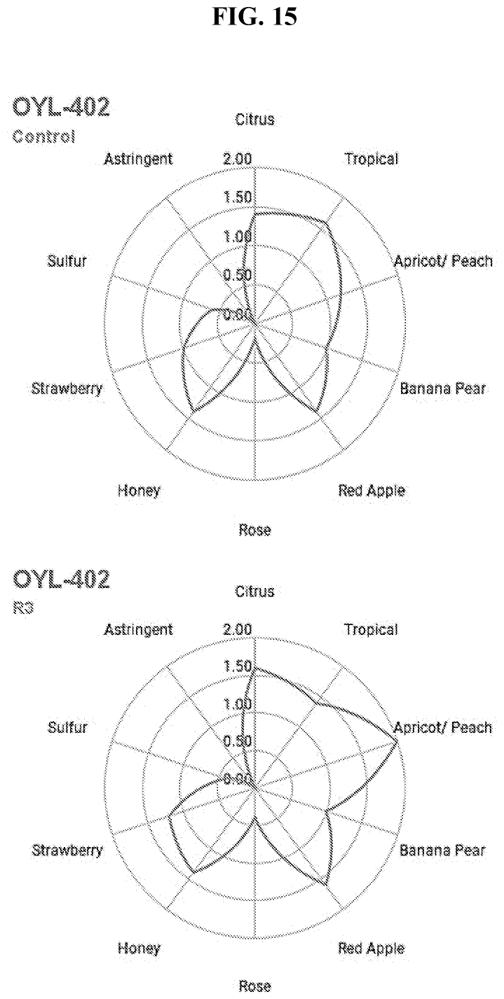

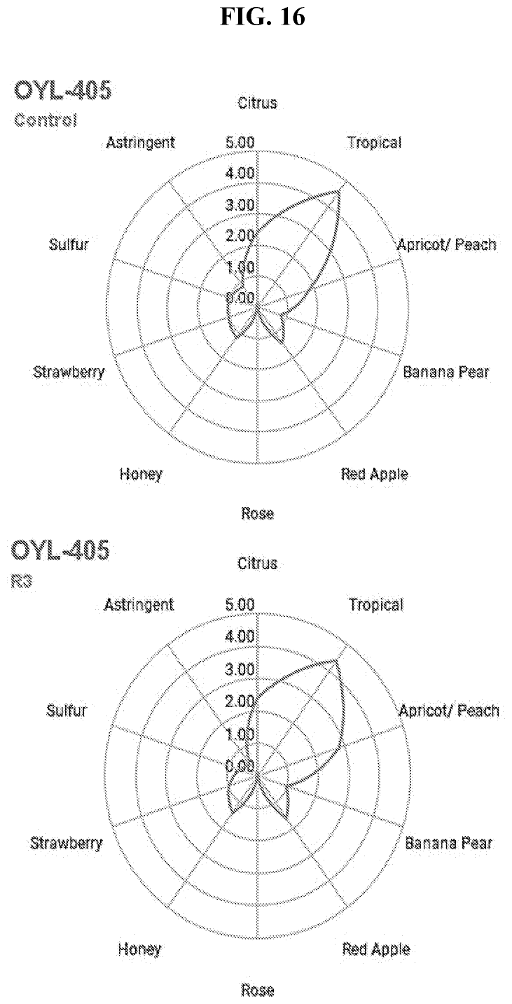

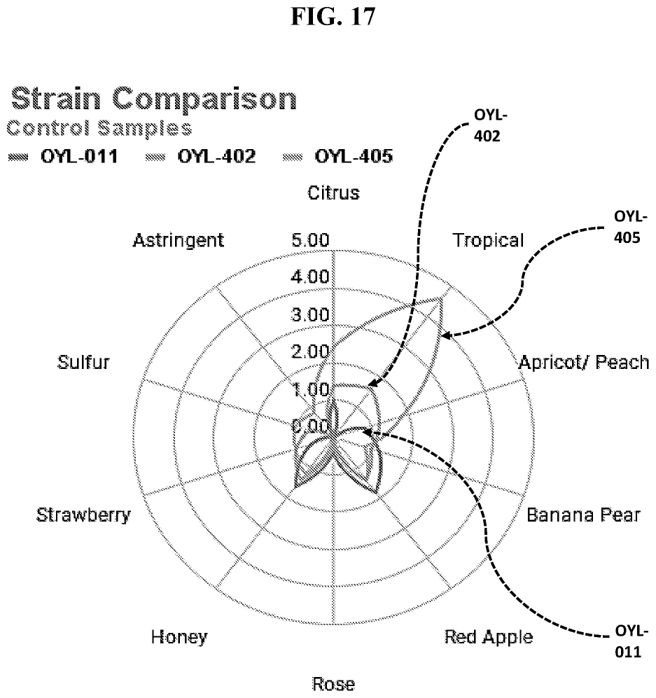

The results are shown in FIGS. 14-18 and Tables 14-15. It is noted that the combined sensory charts are provided at different scales for visual clarity. The results demonstrated that addition of the extract compositions produced a significant sensory impact, particularly when used in conjunction with the OYL-402 strain (FIG. 15). Compared to the control, beer prepared with R3 extract produced substantial shifts in the sensory traits towards apricot/peach and citrus characteristics (FIGS. 15 and 16). The R3 extract also added a light astringency to the beer, particularly when used in conjunction with the OYL-405 (FIG. 16).

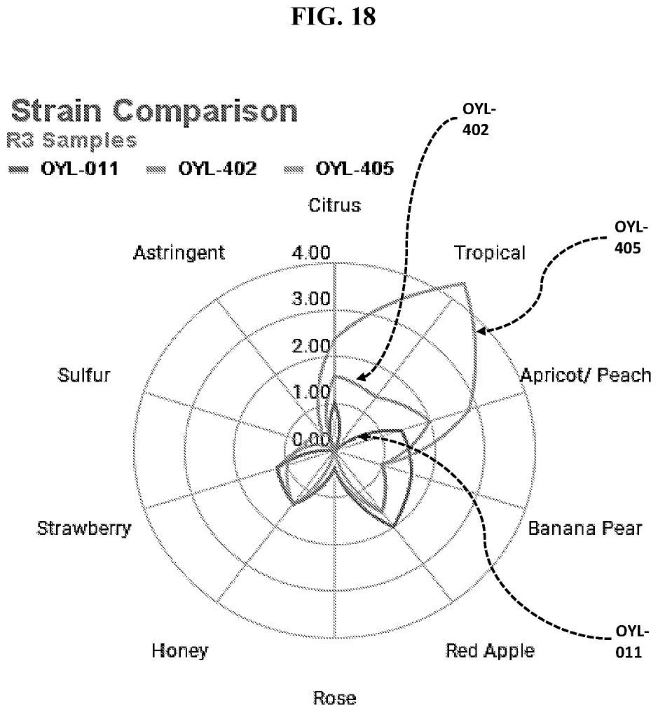

Strain selection affected the overall trajectory of the sensory data. For example, all OYL-405 data was characterised by a rocket shape that sets it apart from the OYL-402 and OYL-011 data (see, e.g., FIG. 18). This was attributed to the tremendous amount of thiol character that the OYL-405 strain provides.

The results suggested that the extract compositions can be used to increase the perceived intensity of pre-existing fruit characters. Favourable aroma characteristics were specifically increased (see, e.g., Table 15). The fermentation temperature was controlled in these trials, and this avoided appearance of fusel alcohol notes. Interestingly, a reduction in fusel alcohol notes appeared to coincide with a reduction in the perceived astringent character.

TABLE 7
Thiol precursors in grape skin extract compositions

| | | Thiol precursors | | | | | |
|---|---|---|---|---|---|---|---|
| | | | cysteinyl- | γ-glutamyl- | gluta- | | gluta- |
| | | cysteine- | glycine- | cysteine- | thione- | cysteine- | thione- |
| | | 3MH | 3MH | 3MH | 3MH | 4MMP | 4MMP |
| | | C3MH | CG3MH | GC3MH | G3MH | C4MMP | G4MMP |
| Sample | Material tested | (μg/g) | (μg/g) | (μg/g) | (μg/g) | (μg/g) | (μg/g) |
| 211205 | Grape skin extract | 2.4330 | 0.2902 | 0.1766 | 1.2979 | 0.0288 | nd |
| | powder form | | | | | | |
| 211205 | Grape skin concen- | 2.1311 | 0.2489 | 0.1651 | 1.2038 | 0.0243 | nd |
| | trated liquid extract | | | | | | |

TABLE 8
Volatile thiols and thiol precursors in beer and wort samples

| | | | | Volatile thiols (free thiols) | | | |
|---|---|---|---|---|---|---|---|
| | | | | | 3-sulfanyl- | 3-sulfanyl- | 4-methyl- |
| | | | | 3-sulfanyl- | 4-methyl- | hexan-1-ol | 4-sulfanyl- |
| | | | | hexan-1-ol | pentan-1-ol | Acetate | pentan-2-one |
| | | | | 3SH | 3S4MPol | 3SHA | 4SMP |
| | | Material | | (3MH) | (3M4MP) | (3MHA) | (4MMP) |
| Sample | Treatment | tested | Quantity | (ng/L) | (ng/L) | (ng/L) | (ng/L) |
| 211205 | 40-2 | beer | 45 mL | 410 | nd | 12 | nd |
| 211205 | 40-16 | beer | 45 mL | 486 | nd | 11 | nd |
| 211205 | 40-18 | beer | 45 mL | 500 | nd | 12 | nd |
| 211205 | 40 control | wort | 45 mL | | | | |
| | wort | | | | | | |
| 211205 | 40 Ex.-D | wort | 45 mL | | | | |
| 211205 | 40 Ex.-L | wort | 45 mL | | | | |

| | Volatile thiols | Thiol precursors | | | | |
|---|---|---|---|---|---|---|
| | (free thiols) | cysteinyl- | γ-glutamyl- | gluta- | | gluta- |
| | cysteine- | glycine- | cysteine- | thione- | cysteine- | thione- |
| | 3MH | 3MH | 3MH | 3MH | 4MMP | 4MMP |
| | C3MH | CG3MH | GC3MH | G3MH | C4MMP | G4MMP |
| Sample | (μg/L) | (μg/L) | (μg/L) | (μg/L) | (μg/L) | (μg/L) |
| 211205 | | | | | | |
| 211205 | | | | | | |
| 211205 | | | | | | |
| 211205 | 2 | 68 | 9 | 49 | nd | nd |
| 211205 | 9 | 68 | 8 | 50 | nd | nd |
| 211205 | 10 | 75 | 9 | 54 | nd | nd |

TABLE 11
Analysis of fractions for thiol precursors and polyphenolics (dry basis)

| | | | | | Cys-3SH + | Cat | Cat | |
|---|---|---|---|---|---|---|---|---|
| Sample | Solids | Cys-3SH | GSH-3SH | Cys-3SH | GSH-3SH | Dimer | Monomer | |
| [g/100 g] | [Peak] | [Peak] | [μg/g] | [μg/g] | [Peak] | [Peak] | [Peak] | Phenolics |
| 02MF50CR1 | 5.28 | 223 | 130 | 1.732 | 2.742 | 42 | 163 | 478 |
| 02MF50CR2 | 5.42 | 216 | 173 | 1.634 | 2.942 | 2722 | 2739 | 7141 |
| 02MF70CR3 | 5.96 | 231 | 185 | 1.588 | 2.859 | 2670 | 2239 | 6612 |
| 02MF70CR4 | 3.99 | 170 | 140 | 1.748 | 3.187 | 2087 | 2044 | 5385 |
| 02RO50CR1 | 12.93 | 474 | 293 | 1.502 | 2.431 | 63 | 224 | 872 |
| 02RO50CR2 | 8.77 | 582 | 401 | 2.720 | 4.594 | 491 | 752 | 2311 |
| 02RO70CR3 | 10.70 | 474 | 361 | 1.816 | 3.199 | 4842 | 4079 | 12016 |
| Std (01FD1) | | | | 2.433 | 3.126 | | | |
| 02MF50CR1 FD | | | | 1.941 | 2.884 | | | |
| 02RO50CR1 FD | | | | 1.812 | 2.818 | | | |

TABLE 12
Sensory information for beer samples

| | | | Extract | |
|---|---|---|---|---|
| Flask sample | Yeast Strain | Treatment | g/L (ml) | Sensory |
| 1 - Control | OYL-402 | n/a | n/a | Moderate haze. Aroma: Moderate sulphur, grain, fruity esters. Taste: Sulphur that adds a |
| | | | | sharpness, slight tropical note. |
| R1 6 g/L | OYL-402 | R1 | 6 (46.60) | Good clarity. Aroma: very similar to the control with an extra fruitiness. Taste: Very fruity, |
| | | | | more pleasant than the powders, less sulphur and so less sharp than the control. |
| R1 12 g/L | OYL-402 | R1 | 12 (93.2) | Good clarity. Aroma: Fruity, a stale note. Taste: Stronger taste, but initially unpleasant. |
| | | | | Some tropical notes, but a stale undertone and slight bitterness. |
| R2 6 g/L | OYL-402 | R2 | 6 (68.42) | Good clarity. Aroma: Fruity, not lending itself to tropical as much as the control. Taste: |
| | | | | Pleasantly fruity, similar to R1, good tropical character, more S. blanc notes than the |
| | | | | others, more intense than the control (pleasantly). Slight raisin undertones. |
| R3 6 g/L | OYL-402 | R3 | 6 (56.07) | Good clarity. Aroma: Fruity, hint of berry along with tropical. Taste: slightly drying, good |
| | | | | tropical character, more intense than the control (pleasantly), mild acidity. |
| R4 6 g/L | OYL-402 | R4 | 6 (150.38) | Good clarity. Aroma: Very, very close to the control but without the sulphur note, |
| | | | | enhanced tropical notes (less masking). Taste: Fruity, but slightly drying with a lightly |
| | | | | bitter finish. |

TABLE 13-1
Thiol precursor and pH levels in wort samples

| Flask | | Starting | Final | C3MH | CG3MH | GC3MH | G3MH | C4MMP | G4MMP |
|---|---|---|---|---|---|---|---|---|---|
| Sample | | pH | pH | (μg/L) | (μg/L) | (μg/L) | (μg/L) | (μg/L) | (μg/L) |
| 1 - Control | | 5.43 | 4.51 | 4 | 86.7 | 10.2 | 36.9 | nd | nd |
| R1 6 | g/L | 5.08 | 4.47 | 15.4 | 90 | 14 | 45.3 | nd | nd |
| R1 12 | g/L | 4.86 | 4.41 | 30 | 93.8 | 15.3 | 59.2 | nd | nd |
| R2 6 | g/L | 5.07 | 4.45 | 15.4 | 88.8 | 12.5 | 49.6 | nd | nd |
| R3 6 | g/L | 5.1 | 4.46 | 16.9 | 87.9 | 12.9 | 45.8 | nd | nd |
| R4 6 | g/L | 4.89 | 4.36 | 18.9 | 81.9 | 12 | 50.8 | nd | nd |

TABLE 13-2
Volatile thiol levels and specific gravity in beer samples

| | | Starting | Final | 3SH | 3S4MPol | 3SHA | 4SMP |
|---|---|---|---|---|---|---|---|
| | | Gravity | Gravity | (3MH) | (3M4MP) | (3MHA) | (4MMP) |
| Flask Sample | | (°P) | (°P) | (ng/L) | (ng/L) | (ng/L) | (ng/L) |
| 1 - Control | | 14.7 | 2.9 | 566 | nd | 42 | 3.3 |
| R1 6 | g/L | 14.8 | 2.7 | 1120 | nd | 22 | 6.9 |
| R1 12 | g/L | 14.6 | 2.4 | 2078 | nd | 45 | 4.6 |
| R2 6 | g/L | 14.3 | 2.7 | 1335 | nd | 30 | 3.1 |
| R3 6 | g/L | 14.5 | 2.7 | 1032 | nd | 22 | 4 |
| R4 6 | g/L | 13.2 | 2.4 | 719 | nd | 18 | nd |

TABLE 14
Measured parameters for beer samples

| | | | Extract | | | Starting | Final | |
|---|---|---|---|---|---|---|---|---|
| | | Wort Tube | (WP addn | Starting | Final | Gravity | Gravity | ABV |
| Sample | Strain | Label | 4.3 g/L) | pH | pH | (°P) | (°P) | (% v/v) |
| 1 - Control | OYL-011 | Control Wort | n/a | 5.4 | 4.67 | 15.3 | 3 | 6.65 |
| R3 + OYL-011 | OYL-011 | R3 Wort | R3 | 5.4 | 4.67 | 15.5 | 3 | 6.65 |
| 2 - Control | OYL-402 | n/a | n/a | 5.4 | 4.58 | 15.3 | 3 | 6.65 |
| R3 + OYL-402 | OYL-402 | n/a | R3 | 5.4 | 4.63 | 15.5 | 3 | 6.65 |
| 3 - Control | OYL-405 | n/a | n/a | 5.4 | 4.65 | 15.3 | 2.7 | 6.81 |
| R3 + OYL-405 | OYL-405 | n/a | R3 | 5.4 | 4.67 | 15.5 | 3 | 6.65 |

TABLE 15
Sensory scores for beer samples

| | Sample | | | | | |
|---|---|---|---|---|---|---|
| Aroma/Taste | 1 - Control | R3 + OYL-011 | 2 - Control | R3 + OYL-402 | 3 - Control | R3 + OYL-405 |
| Citrus | 1.00 | 1.00 | 1.40 | 1.60 | 2.40 | 2.40 |
| Passionfruit/ | 0.00 | 0.00 | 1.60 | 1.40 | 4.60 | 4.40 |
| Guava/Tropical | | | | | | |
| Apricot/Peach | 0.80 | 1.40 | 1.20 | 2.00 | 1.80 | 2.80 |
| Banana/Pear | 1.20 | 1.60 | 1.00 | 1.00 | 0.80 | 1.00 |
| Red Apple | 1.80 | 2.00 | 1.40 | 1.60 | 1.40 | 1.60 |
| Rose | 0.40 | 0.40 | 0.20 | 0.40 | 0.00 | 0.00 |
| Honey | 1.60 | 1.40 | 1.40 | 1.40 | 1.20 | 1.40 |
| Strawberry | 0.60 | 1.20 | 1.00 | 1.20 | 1.00 | 1.00 |
| Sulphur | 0.00 | 0.00 | 0.60 | 0.40 | 1.00 | 0.60 |
| Astringent | 0.00 | 0.00 | 0.00 | 0.00 | 0.80 | 0.40 |

Persons of ordinary skill can utilise the disclosures and teachings herein to produce other embodiments and variations without undue experimentation. All such embodiments and variations are considered to be part of this disclosure.

Accordingly, one of ordinary skill in the art will readily appreciate from the disclosure that later modifications, substitutions, and/or variations performing substantially the same function or achieving substantially the same result as embodiments described herein may be utilised according to such related embodiments. Thus, this disclosure is intended to encompass, within its scope, the modifications, substitutions, and variations to processes, manufactures, compositions of matter, compounds, means, methods, and/or steps set out herein.

In this specification, where reference has been made to external sources of information, including patent specifications and other documents, this is generally for the purpose of providing a context for discussing the features of this disclosure. Unless stated otherwise, reference to such sources of information is not to be construed, in any jurisdiction, as an admission that such sources of information are prior art or form part of the common general knowledge in the art.

The description herein may contain subject matter that falls outside of the scope of the claimed invention. This subject matter is included to aid understanding of the invention.

### REFERENCES

Anfang, N, Brajkovich, M, Goddard, M R. Co-fermentation with Pichia kluyveri increases varietal thiol concentrations in Sauvignon Blanc. Australian Journal of Grape Wine Research 15(1), 1-8, 2009.
Brajkovich, M, Peron, G, Lund, C M, Dykes, S I, Kilmartin, P A, Nicolau, L. Effect of screwcap and cork closures on S02 levels and aromas in a Sauvignon Blanc wine. Journal of Agricultural and Food Chemistry 53, 10006-10011, 2005.
Coetzee, C D T, du Toit, W J. A comprehensive review on Sauvignon blanc aroma with a focus on certain positive volatile thiols. Food Research International 45, 287-298, 2012.
Darriet, P, Tominaga, T, Lavigne, V, Boidron, J-N, Dubourdieu, D. Identification of a powerful aromatic component of Vitis vinifera L. var. Sauvignon wines: 4-mercapto-4-methylpentan-2-one. Flavour and Fragrance Journal 10(6), 385-392, 1995.
de Torres, C, Schumacher, R, Alañón, M E, Pérez-Coello, M S, Díaz-Maroto, M C. Freeze-dried grape skins by-products to enhance the quality of white wines from neutral grape varieties. Food Research International 69, 97-105, 2015.
Du Plessis, C S, Augustyn, O P H. Initial Study on the Guava Aroma of Chenin Blanc and Colombar Wines. South African Journal of Enology and Viticulture 2, 101, 1981.
Escalante, W D E. Perspectives and uses of non-Saccharomyces yeasts in fermented beverages. Frontiers and New Trends in the Science of Fermented Food and Beverages, IntechOpen, DOI: http://dx.doi.org/10.5772/intechopen.81868
Holt, S, Miks, M H, de Carvalho, B T, Foulquie-Moreno, M R, Thevelein, J M. The molecular biology of fruity and floral aromas in beer and other alcoholic beverages. FEMS Microbiology Reviews 43, 193-222, 2019.
Jelley, R E, Herbst-Johnstone, M, Klaere, S, Pilkington, L I, Grose, C, Martin, D, Barker, D, Fedrizzi, B, Optimization of ecofriendly extraction of bioactive monomeric phenolics and useful flavor precursors from grape waste. ACS Sustainable Chemical Engineering 4, 5060-5067, 2016.
Jelley, R E, Deed, R C, Barker, D, Parish-Virtue, K, Fedrizzi, B. Fermentation of Sauvignon blanc grape marc extract yields important wine aroma 3-sulfanylhexan-1-ol (3SH). LWT—Food Science and Technology 131 109653, 2020.
Lund, C M T, Benkwitz, F, Wohler, M W, Triggs, C M, Gardner, R, Heymann, H, Nicolau, L. New Zealand Sauvignon Blanc Distinct Flavor Characteristics: Sensory, Chemical, and Consumer Aspects. American Journal of Enology and Viticulture 60, 1-12, 2009.
Maggu, M, Winz, R, Kilmartin, P A, Trought, M C T, Nicolau, L. effect of skin contact and pressure on the composition of sauvignon blanc must. Journal of Agricultural and Food Chemistry 55, 10281-10288, 2007.
Ribéreau-Gayon, P, Glories, Y, Maujean, A, Dubourdieu, D. Handbook of Enology: The chemistry of wine stabilization and treatments. John Wiley & Sons Ltd, Chicheste, 2006.
Robinson, J (ed) The Oxford Companion to Wine, Third Edition, Oxford University Press, 2006.
Roland, A, Schneider, R, Razungles, A, Cavelier, F. Varietal thiols in wine: discovery, analysis, and applications. Chemical Reviews 111, 7355-7376, 2011.
Spanos, G A and Wrolstad, R E. Influence of processing and storage on the phenolic composition of Thompson Seedless grape juice. Journal of Agricultural and Food Chemistry 38(7): 1565-1571, 1990.
Takoi, K, Degueil, M, Shinkaruk, S, Thibon, C, Maeda, K, Ito, K, Bennetau, B, Dubourdieu, D, Tominaga, T. Identification and characteristics of new volatile thiols derived from the Hop (Humulus luplus L.) cultivar Nelson Sauvin. Journal of Agricultural and Food Chemistry 57, 2493-2502, 2009.
Tominaga, T, Dubourdieu, D. A New Type of Flavor Precursors in Vitis vinifera L cv. Sauvignon Blanc-S-Cysteine Conjugates. Journal of Agricultural and Food Chemistry 46, 5215-5219, 1998.
Tominaga, T, Furrer, A, Henry, R, Dubourdieu, D. Identification of new volatile thiols in the aroma of Vitis vinifera L. var. Sauvignon Blanc wines Flavour and Fragrance Journal 13, 159-162, 1998.
Tominaga, T, Darriet, P, Dubourdieu, D. Identification de l'acétate de 3-mercaptohexanol, composé à forte odeur de buis, intervenant dans l'arôme des vins de Sauvignon Vitis 35, 207-210, 1996.

1.-54. (canceled)

55. A method for preparing a composition comprising a grape skin extract, the method comprising:
(i) obtaining skin of white wine grapes, such skin having flesh and juice components substantially removed,
(ii) freezing the skin of the white wine grapes,
(iii) thawing the skin of the white wine grapes,
(iv) incubating the skin of the white wine grapes in aqueous solution at about 45° C. to about 75° C.,
(v) obtaining a liquid extract produced by the incubating of the skin of the white wine grapes,
(vi) optionally removing sugars from the liquid extract,
(vii) optionally drying the liquid extract to produce a substantially dried product.

56. The method of claim 55, wherein:
(a) the white wine grapes are aromatic white wine grapes;
(b) the white wine grapes are selected from the group consisting of: white wine grapes from New Zealand, Australia, South Africa, South America, and any combination thereof; and/or
(c) the white wine grapes are selected from the group consisting of: Sauvignon Blanc, Albariño, Chenin Blanc, Colombard, Friulano, Tocai, Grüner Veltliner, Traminer, Verdicchio, Verdejo, Vermentino, Scheurebe, Maccabeo, Gewürztraminer, Riesling, Muscat, Petit Manseng, Pinot Gris, and Tokay grapes, and any combination thereof.

57. The method of claim 55, wherein:
(a) the white wine grapes comprise Sauvignon Blanc grapes;
(b) the white wine grapes consist essentially of Sauvignon Blanc grapes, or
(c) the white wine grapes are Sauvignon Blanc grapes.

58. The method of claim 55, wherein:
(a) the substantially removing of (i) comprises crushing or pressing;
(b) the substantially removing of (i) comprises pressing and the pressing is performed by a pneumatic press or a basket press;
(c) the substantially removing of (i) comprises pressing and the pressing is performed with a press setting ranging from about 600 to about 800 litres per tonne; or
(d) the substantially removing of (i) comprises pressing and the pressing is performed with a press setting ranging from about 650 to about 720 litres per tonne.

59. The method of claim 55, wherein:
(a) the method includes addition of at least one antioxidant or preservative before or after (i);
(b) the method includes addition of a sulphite before or after (i); or
(c) the method includes addition of ascorbic acid before or after (i).

60. The method of claim 55, which comprises one or more of:
(a) the aqueous solution of (i) is water or a solution consisting essentially of water;
(b) the freezing of (ii) comprises blast freezing;
(c) the freezing of (ii) comprises cryomaceration;
(d) the incubating of (iv) comprises incubation at about 50° C. or higher, or about 55° C. or higher, or about 60° C. or higher, or about 65° C. or higher, or about 70° C. or higher;
(e) the removing of (vi) is by column absorption or desorption;
(f) the removing of (vi) is by fermentation; and
(g) the drying of (iv) comprises freeze drying.

61. The method of claim 55, which further comprises one or more of:
(a) contacting the liquid extract of (v) with further grape skin processed by any of (i)-(iii) to obtain a liquid extract that is further concentrated;
(b) pasteurizing the liquid extract of (v);
(c) milling the dried product of (vii) to obtain a milled product;
(d) formulating the dried product of (vii) to obtain a pellet product;
(e) formulating the dried product of (vii) to obtain a flake product or a powder product;
(e) mixing the dried product of (vii) with one or more liquids to obtain a liquid or semi-liquid product; and
(f) formulating the composition to include one or more hops or hops components.

62. The method of claim 61, wherein:
(a) the one or more hops are selected from the group consisting of: American, Australian, English, German, New Zealand, and South African hops, and any combination thereof;
(b) the one or more hops are selected from the group consisting of: Nelson Sauvin hops, Hallertau Blanc hops, Tomahawk hops, Simcoe hops, Summit hops, Cascade hops, and any combination thereof;
(c) the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof; or
(d) the one or more hops components are selected from the group consisting of lupulin, hops terpenes, and hops esters.

63. A composition comprising a grape skin extract, the composition being prepared according to the method of claim 55.

64. The composition of claim 63, wherein:
(a) the composition comprises a milled form;
(b) the composition comprises a flake form or a powder form;
(c) the composition comprises a pellet form; or
(d) the composition comprises a liquid form or a semi-liquid form.

65. The composition of claim 63, which comprises one or more of:
(a) the composition further comprises one or more yeast;
(b) the composition further comprises one or more bacteria;
(c) the composition further comprises one or more enzymes; and
(d) the composition further comprises one or more hops or hops components.

66. The composition of claim 65, wherein:
(a) the one or more yeast are selected from the group consisting of: Saccharomyces, Brettanomyces, Kloeckera, Candida, Hanseniaspora, Pichia yeast, and any combination thereof;
(b) the one or more yeast include a Pichia kluyveri yeast;
(c) the one or more yeast include a Saccharomyces cerevisiae yeast;
(d) the one or more yeast include a brewer's yeast;
(e) the one or more yeast include yeast used for conversion of thiol precursors into free thiols;
(f) the one or more bacteria are selected from the group consisting of: Lactobacillus bacteria, Pediococcus bacteria, and any combination thereof;
(g) the one or more bacteria are selected from the group consisting of: Lactobacillus delbrueckii, Lactobacillus plantarum, Pediococcus damnosus, and any combination thereof;
(h) the one or more bacteria include a bacteria used for lactic acid production;
(i) the one or more enzymes include β-lyase;
(j) the one or more enzymes include β-glucosidase;
(k) the one or more hops include cryogenically prepared hops;
(l) the one or more hops components include lupulin;
(m) the one or more hops components include hops terpenes; or
(n) the one or more hops components include hops esters.

67. The composition of claim 65, wherein:
(a) the one or more hops are selected from the group consisting of: American, Australian, English, German, New Zealand, and South African hops, and any combination thereof,
(b) wherein the one or more hops are selected from the group consisting of: Nelson Sauvin hops, Hallertau Blanc hops, Tomahawk hops, Simcoe hops, Summit hops, Cascade hops, and any combination thereof, and/or
(c) the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

68. A method of making a fermented beverage, comprising:
introducing a composition comprising a grape skin extract during a fermentation and/or prior to initiation of a fermentation,
wherein the composition comprising the grape skin extract is prepared according to the method of claim 55, and
carrying out the fermentation, thereby obtaining the fermented beverage.

69. The method of claim 68, wherein:
(a) the method includes introducing the composition post-fermentation;
(b) the method includes introducing one or more yeast during the fermentation and/or prior to initiation of the fermentation,
(c) the method includes introducing one or more bacteria during the fermentation and/or prior to initiation of the fermentation,
(d) the method includes introducing one or more enzymes during the fermentation and/or prior to initiation of the fermentation, and/or
(e) the method includes introducing one or more hops or hops components during the fermentation, prior to initiation of the fermentation, and/or after the fermentation.

70. The method of claim 68, wherein:
(a) the composition comprises a milled form;
(b) the composition comprises a flake form or a powder form;
(c) the composition comprises a pellet form; or
(d) the composition comprises a liquid form or a semi-liquid form.

71. The method of claim 68, wherein:
(a) the composition further comprises one or more yeast;
(b) the composition further comprises one or more bacteria;
(c) the composition further comprises one or more enzymes; or
(d) the composition further comprises one or more hops or hops components.

72. The method of claim 71, wherein:
(a) the one or more yeast are selected from the group consisting of: Saccharomyces, Brettanomyces, Kloeckera, Candida, Hanseniaspora, Pichia yeast, and any combination thereof;
(b) the one or more yeast include a Pichia kluyveri yeast;
(c) the one or more yeast include a Saccharomyces cerevisiae yeast;
(d) the one or more yeast include a brewer's yeast;
(e) the one or more yeast include yeast used for conversion of thiol precursors into free thiols;
(f) the one or more bacteria are selected from the group consisting of: Lactobacillus bacteria, Pediococcus bacteria, and any combination thereof;
(g) the one or more bacteria are selected from the group consisting of: Lactobacillus delbrueckii, Lactobacillus plantarum, Pediococcus damnosus, and combination thereof;
(h) the one or more bacteria include a bacteria used for lactic acid production;
(i) the one or more enzymes include β-lyase;
(j) the one or more enzymes include β-glucosidase;
(k) the one or more hops include cryogenically prepared hops;
(l) the one hops components include lupulin;
(m) the one or more hops components include hops terpenes; or
(n) the one or more hops components include hops esters.

73. The method of claim 71, wherein:
(a) the one or more hops are selected from the group consisting of: American, Australian, English, German, New Zealand, and South African hops, and any combination thereof,
(b) wherein the one or more hops are selected from the group consisting of: Nelson Sauvin hops, Hallertau Blanc hops, Tomahawk hops, Simcoe hops, Summit hops, Cascade hops, and any combination thereof, and/or
(c) wherein the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

74. The method of claim 68, wherein:
(a) the fermented beverage is an alcoholic, non-alcoholic, or reduced-alcohol beverage;
(b) the fermented beverage is a beer, fruit beer, beer-wine hybrid, cider, or kombucha;
(c) the fermented beverage is selected from the group consisting of: barley or other cereal beers, maize beers, millet beers, oat beers, rice beers, rye beers, sorghum beers, and wheat beers;
(d) the fermented beverage is selected from the group consisting of: ales, lagers, pilsners, porters, saisons, and stouts;
(e) the fermented beverage is a India Pale Ale (IPA) beer or a New England IPA beer; or
(f) the fermented beverage is a hazy IPA beer.

75. A fermented beverage prepared using the method according to claim 68.

76. The fermented beverage of claim 75, wherein:
(a) the fermented beverage is an alcoholic, non-alcoholic, or reduced-alcohol beverage;
(b) the fermented beverage is a beer, fruit beer, beer-wine hybrid, cider, or kombucha;
(c) the fermented beverage is selected from the group consisting of: barley or other cereal beers, maize beers, millet beers, oat beers, rice beers, rye beers, sorghum beers, and wheat beers;
(d) the fermented beverage is selected from the group consisting of: ales, lagers, pilsners, porters, saisons, and stouts;
(e) the fermented beverage is a India Pale Ale (IPA) beer or a New England IPA beer; or
(f) the fermented beverage is a hazy IPA beer.
